(19) United States
(12) Patent Application Publication
(10) Pub. No.: US 2023/0212581 A1
(43) Pub. Date: Jul. 6, 2023

(54) COMPOSITIONS AND METHODS OF INDUCING RNAI OR TYPE I IFN COMPETENT CELLS AND USES THEREOF

(71) Applicant: WILFRID LAURIER UNIVERSITY, Waterloo (CA)

(72) Inventor: Stephanie Dewitte-Orr, Waterloo (CA)

(21) Appl. No.: 17/927,840

(22) PCT Filed: May 25, 2021
(86) PCT No.: PCT/CA2021/050703

§ 371 Date: Nov. 25, 2022

Related U.S. Application Data
(60) Provisional application No. 63/029,632, filed on May 25, 2020.

(51) Int. Cl. C12N 15/113; G01N 33/50; A61P 31/00

(52) U.S. Cl. CPC C12N 15/1131; G01N 33/5023; C12N 15/1136; A61P 31/00; C12N 2310/14

(57) ABSTRACT

Provided is a double stranded RNA (dsRNA) compound comprising a guide strand and a passenger strand, the guide strand and the passenger strand each having a length of at least 300 basepairs (bp), the guide strand comprising a segment complementary to a target nucleic acid sequence of a target gene transcript. Also provided are methods of silencing a target gene transcript in a vertebrate cell or subject, of treating a pathogen infection in a subject, and of reducing replication or infectivity of a pathogen infection in the vertebrate cell or subject, respectively, comprising administering to the subject or cell a dsRNA compound, vector, conjugate or composition herein disclosed.

### RELATED APPLICATION

This is a Patent Cooperation Treaty Application which claims the benefit of 35 U.S.C. § 119 based on the priority of U.S. Provisional Patent Application No. 63/029,632, filed May 25, 2020 which is herein incorporated in its entirety by reference.

### INCORPORATION OF SEQUENCE LISTING

A computer readable form of the Sequence Listing “P58544PC00 Sequence Listing_ST25” (8,399 bytes), submitted via EFS-WEB and created on May 25, 2021, is herein incorporated by reference.

### FIELD

The present disclosure relates to compositions and methods of for decreasing expression of a target gene or target genes using ribonucleic acid interference (RNAi). The disclosure also in particular relates to compositions and methods that can be modulated to evade or induce a type I interferon (IFN) response in a cell or host.

### BACKGROUND

Long double-stranded (ds) RNA (40 bp or more) is not found in healthy cells and is made by almost all viruses sometime during their replicative cycle (Jacobs-1996). These long dsRNA compounds are sensed by pattern recognition receptors (PRRs) on the surface (class A scavenger receptors), endosome (Toll like receptor 3) and cytoplasm (RIG-1-like receptors and cytoplasmic DNA sensors) of cells (DeWitte-Orr-2010b). Once activated the PRRs recruit adapter proteins, which activate signaling cascades that culminate in the increased expression of type I interferons (IFNs). These IFNs act in an autocrine and paracrine fashion through their cognate receptor and downstream signaling cascade to induce the expression of IFN stimulated genes (ISGs) (DeWitte-Orr-2010b). ISGs accumulate in the infected cell and neighboring uninfected cells to establish an ‘antiviral state’ where the cell is refractive to virus infection. The IFN pathway is present in vertebrates, which have the ability to produce type I IFN.

RNA interference (RNAi) was first characterized in invertebrates and plants as an antiviral innate immune response. It is a natural antiviral defense mechanism to degrade viral RNA by virus-induced gene silencing (Abedini et al., 2018). Studies showed synthetic double stranded RNA (dsRNA) induces sequence-specific degradation of mRNA, and resulting in gene silencing (Fire et al., 1998). In plants and invertebrates long dsRNA (>40 bp) shuts down specific gene expression via the RNAi pathway (Buchon-2005). The type Ill ribonuclease Dicer binds long dsRNA compounds, cleaves it into small dsRNA compounds (short interfering (si) RNA) that are loaded into RISC and used to find complementary mRNA sequences to bind and either inhibit its translation by steric interference or by mRNA degradation (Meister-2004). Plants and invertebrates do not have IFN production.

IFN induces global shut down of both transcription and translation, which masks or inhibits RNAi's sequence specific effects (Maillard-2016). It has been shown that long dsRNA (at least 30 bp) can trigger RNAi in mammalian cells that are IFN-deficient (Maillard-2016).

DsRNA length does affect IFN production with longer molecules inducing a stronger IFN response (DeWitte-Orr et al., 2010).

US20130330824 discloses a method for the use of long dsRNA for gene silencing in mammalian cells using bacteria, the method comprising transforming long dsRNA into bacteria, having the long dsRNA processed into a mixture of smaller RNA duplexes and released into the cytoplasm of the target cells.

WO2017136895 discloses constructs producing dsRNA over 400 base pairs long for targeting viruses in plants, which do not have an IFN response.

US20170253881 discloses the introduction of long dsRNA (over 30 base pairs) for gene silencing in cells, using a vector, named pDECAP, to express long double-strand RNA from an RNA polymerase II (Pol II) promoter.

WO2018146557 discloses long dsRNA duplexes transfected into cells as capable of inhibiting the expression of two different target mRNA sequences.

U.S. Pat. No. 8,299,042 discloses methods of post-transcriptional gene silencing which involve the use of a first dsRNA having substantial sequence identity to a target nucleic acid and a short, second dsRNA which inhibits dsRNA-mediated toxicity.

### SUMMARY

Provided herein in various aspects are compositions comprising dsRNA compounds of greater than 300 base pairs, and methods for their use for silencing a target gene transcript. In some embodiments, the compositions and methods can be used to evade or induce a type I interferon (IFN) response in target cells.

As demonstrated herein, the inventors have determined methods and compositions comprising for example long dsRNA to decrease target gene expression without induction of type I IFN which ablates RNA interference. As described herein long dsRNA compounds and methods have been developed that can silence a target gene transcript via RNAi in vertebrate cells that are IFN competent. The inventors have found for example that by modulating the dosage and/or length of long dsRNA compounds they can avoid or induce induction of type I IFN.

A first aspect includes a double stranded RNA (dsRNA) compound comprising a guide strand and a passenger strand, the guide strand and the passenger strand each having a length of at least 300 basepairs (bp), and the guide strand comprising a segment complementary to a target nucleic acid sequence of a target gene transcript.

Another aspect relates to the dsRNA compound described herein for use in silencing a target gene transcript in a vertebrate cell or subject comprising the target nucleic acid sequence, and/or for use in reducing replication or infectivity of a pathogen infection in the vertebrate cell or subject, respectively.

Another aspect relates to the use of the dsRNA compound described herein for use in targeting a mixed cell population by silencing a target gene transcript in a first vertebrate cell type comprising the target nucleic acid sequence and inducing a type I interferon (IFN) response in a second vertebrate cell type, optionally wherein the cell population is in a vertebrate subject.

Another aspect is a conjugate comprising the dsRNA compound described herein and one or more polypeptide, such as an antibody, optionally a single chain antibody or a binding fragment, or a label.

Another aspect includes a vector comprising an expression cassette encoding one or more transcript(s) that can form the dsRNA compound described herein.

Another aspect includes a composition comprising the dsRNA compound, conjugate, or vector described herein.

Another aspect includes a method of silencing a target gene transcript in a vertebrate cell or subject, the method comprising administering to the cell or subject an amount of a dsRNA compound, vector, conjugate, or composition described herein. The target gene transcript can for example be a cancer promoting gene transcript. Also provided is a method of treating a cancer comprising administering to a subject in need thereof an amount of the dsRNA compound, vector, conjugate or composition described herein, wherein the target gene transcript is the product (e.g. gene transcript) of a gene involved in cancer.

Another aspect includes a method for reducing replication or infectivity of a pathogen infection in the vertebrate cell or subject, respectively, wherein the target nucleic acid sequence corresponds to a pathogen gene transcript sequence, such as a viral gene transcript sequence.

Another aspect includes a method of treating a pathogen infection, the method comprising administering to a subject in need thereof an amount of the dsRNA compound, vector, conjugate or composition described herein, wherein the target gene transcript is a pathogen gene transcript, such as a viral gene transcript.

Another aspect is a use of the dsRNA compound, vector, conjugate or composition herein described for silencing a target gene transcript in a vertebrate cell or subject, for reducing replication or infectivity of a pathogen infection such as a viral infection in the cell or subject, treating a pathogen infection in a subject in need thereof and/or for the preparation of a medicament.

### BRIEF DESCRIPTION OF DRAWINGS

An embodiment of the present disclosure will now be described in relation to the drawings in which:

FIG. 1A-F is a series of graphs assessing the viability of cell lines when treated with long dsRNA (700 bp). To ensure that exposure to dsRNA will not result in decreased cellular survival, five cell lines were treated with concentrations of dsRNA (ranging from 3.13 to 800 ng/mL) and viability was assessed after 30 hours using an Alamar Blue/CFDA-AM assay. Survival was not negatively influenced by the treatment, but higher concentrations of dsRNA enhanced metabolic activity (as indicated by Alamar Blue). The cell lines assessed included the THF cells (FIG. 1A, n=3), the M14 melanoma cell line (FIG. 1B, n=3), the MRC5 lung fibroblast cell line (FIG. 1C, n=3), the SNB75 glioblastoma cell line (FIG. 1D, n=3), the BT549 breast cancer cell line (FIG. 1E, n=3), and the HepG2 hepatoblastoma cell line (FIG. 1F, n=3).

FIG. 2A is an image and FIGS. 2B and 2C are of graphs illustrating the effects of dsRNA of different lengths. (FIG. 2A) dsRNA can be produced with different lengths, here lengths of 700 bp, 600 bp, 500 bp, 400 bp, 300 bp, 200 bp and 100 bp are demonstrated. (FIG. 2B) telomerase-immortalized human fibroblasts (THF) cells (50,000 cells/well) and (FIG. 2C) SNB75 cells (50,000 cells/well) were both exposed to 500 ng/mL of 700 bp, 600 bp, 500 bp, 400 bp, 300 bp or 200 bp dsRNA length for 2 hours and were then infected with VSV-GFP (multiplicity of infection (MOI)=0.1). Following 24 hours of infection, supernatants were collected and TCID₅₀ was calculated using HEL-299 cells (n=6 for both cell types). Lengths of 400 bp, 500 bp and 600 bp dsRNA was just as effective as 700 bp lengths for limiting VSV-GFP when using a matched sequence (GFP) in THF cells. In SNB75 cells, lengths of 500 bp and 600 bp were comparable to 700 bp lengths, 400 and 300 bp were not statistically significantly different from controls. Both mCherry and beta-lac are mis-matched control sequences.

FIG. 3A-3C are a series of graphs depicting dsRNAi in HEL 299 cells at doses that do not induce interferon stimulated genes (ISGs). (FIG. 3A-3B) Human Embryonic Lung cells (HEL-299; ATCC CCL-137) were pretreated for 2 hours with 500, 1000, or 1500 ng/ml of either mCherry (mis-matched), or eGFP (matched) sequence specific double-stranded RNA (700 bp in length), or poly I:C (500 ng/ml) in serum free media. Total RNA was extracted by Trizol™ at 24 hours post-treatment and subjected to reverse transcription. Expression of interferon stimulated genes (ISGs) beta actin, CXCL10 (FIG. 3B), and ISG15 (FIG. 3A) was measured via qPCR. ISG15, and CxCL10 expression was normalized to actin. While treatment with poly I:C (500 ng/ml) resulted in significant expression of ISGs, treatment with dsRNA, matched or mis-matched, up to 1500 ng/ml resulted in no induction of ISGs above baseline. (FIG. 3C) Human Embryonic Lung cells (HEL-299; ATCC CCL-137) were pretreated for 2 hours with 1000 ng/ml of either beta-lactamase (mis-matched), or eGFP (matched) sequence specific double-stranded RNA in serum free media. Monolayers were subsequently infected with VSV-GFP (Indiana) at an MOI of 0.1. GFP fluorescence was measured by plate reader when infection of mock-treated cells reached 80% (usually 16-24 hours post-infection). All treated samples were calculated as fold relative to mock treated cells. Results presented are pooled data from 4 biological replicates with at least 4 technical replicates per experiment. Protection was seen with dsRNA matching to sequence encoded by the virus.

FIG. 3D-3G are a series of graphs depicting treating THF and SNB75 cells with long 700 bp dsRNA does not induce Type I interferons or interferon stimulated genes (ISGs). Cells were treated with dsRNA (0.5, 10 ug/mL) or poly IC (10 ug/mL) for 26 hours, after which RNA was extracted by TRIzol™ in accordance with the manufacturer's instructions. To remove any contaminating genomic DNA, RNA samples underwent DNase treatment using the Turbo DNase™ Kit (ThermoFisher) as outlined by the manufacturer. Complementary DNA (cDNA) was synthesized from 500 ng of total RNA using the iScript cDNA Synthesis Mix (Bio-Rad) in accordance to the manufacturer's instructions. Expression of p-actin, IFN-β and CXCL10 was measured via qRT-PCR. IFNβ and CXCL10 expression was normalized to β-actin.

FIG. 4A to 4C are a series of graphs depicting dsRNAi can inhibit viral growth using dsRNA of viral genes. Both telomerase-immortalized human fibroblasts (THF) (FIG. 4A) and SNB75 (FIG. 4B) cells were exposed to 500 ng/mL of 700 bp dsRNA of GFP (positive control), VSV N protein, VSV M protein, mCherry and Beta-lac for 2 h before infected with VSV-GFP (MOI=0.1) for 24 h. The cells were also exposed to a mixture containing 250 ng/mL of VSV N protein and 250 ng/mL of VSV M protein, resulting in a total concentration of 500 ng/mL. Both mCherry and Beta-lac acted as mis-matched sequence negative controls. The dsRNA compounds of viral genes (N and M proteins) were able to induce significant inhibition of VSV-GFP but a 500 ng/mL mixture of the two dsRNA sequences did not appear to have an additive effect. (FIG. 4C) MRC5 cells were treated for 2 h with 700 bp HCoV-229E gene dsRNA of RdRp (RNA-dependent RNA-polymerase), M protein, N protein, Spike protein and the mis-matched negative control mCherry. The cells were then infected with HCoV-229E for 24 h and supernatants were collected to determine the TCID₅₀. Treating with HCoC-229E dsRNA for M protein, N protein and Spike protein were able to significantly reduce the viral numbers, while mCherry and RdRp had no protective effect.

FIG. 5A to 5C are a series of graphs depicting viral growth inhibition of primary Bronchial Epithelial Cells (pBECs) by dsRNAi. Following 2 h of exposure to 700 bp sequence-specific dsRNA (N protein) or 700 bp mis-matched sequence (mCherry), pBECs (obtained from a healthy donor) were infected with VSV-GFP (MOI=0.1) for 24 h and supernatant was collected to complete TCID₅₀ quantification (n=2). The matched dsRNA sequence for VSV N protein stimulated an impressive reduction in VSV-GFP titers (FIG. 5A). pBEC cells were pre-treated for 2 h with 700 bp dsRNA of HCoV-229E M protein (0.5 μg/mL), the mis-matched mCherry sequence (0.5 μg/mL) or with polyinosinic:polycytidylic acid (poly I:C, 50 μg/mL). Poly I:C is a potent interferon inducer and would be anticipated to provide protection from viral infection (positive control). Following pre-treating, the pBECs were infected with HCoV-229E (MOI=0.1) for 24 h and supernatants were collected and used for TCID₅₀ quantification. Both the M protein dsRNA (0.5 μg/mL) and the poly I:C (50 μg/mL) stimulated a reduction in viral numbers (n=6) (FIG. 5B). Using the FuGENE transfection reagent dsRNA was transfected into pBEC cells with 700 bp dsRNA of HCoV-229E M protein sequence (0.5 μg/mL) or the mis-matched mCherry sequence (0.5 μg/mL). Transfected cells were then infected with HCoV-229E (MOI=0.1) for 24 h and supernatants were collected and used for TCID₅₀ quantification. The M protein stimulated a reduction in viral numbers (n=2) (FIG. 5C), similar to what was observed in FIG. 5B where no transfection reagent was used.

FIG. 6 is a graph depicting dsRNA effective at limiting virus VSV-GFP replication 1-5 h prior to infection and at the time of infection. M14 Cells (75,000 cells/well) were exposed to 500 ng/mL of each dsRNA (700 bp each; dsRNA encoding GFP (match) or mCherry (mis-match)) at various times before infection with VSV-GFP (MOI=1). Following 24 hours of infection, supernatants were collected and the TCID₅₀ was calculated using HEL-299 cells (n=3). Significant differences were assessed between mCherry and GFP at each individual timepoint.

FIG. 7A to 7D are a series of graphs depicting the inhibition of viral growth can only be observed with siRNA when transfecting mammalian cells. THF (FIG. 7A) and SNB75 (FIG. 7B) cells were treated (“soak”) with GFP siRNA, long GFP dsRNA (matched) or long mCherry dsRNA (mis-matched) for 2 hours and infected with VSV-GFP (MOI=0.1) for 24 hours before supernatant collection. Inhibition of viral growth was only observed in the long sequence matched (GFP dsRNA) exposure condition (n=6 for both cell lines). When GFP siRNA was transfected into THF (FIG. 7C) and SNB75 (FIG. 7D) cells using lipofectamine, a significant reduction in VSV-GFP viral titers was observed (n=4 for both cell lines). These results show that long dsRNA does not require transfection to induce viral inhibition, unlike siRNA.

FIGS. 8A and 8B are a series of graphs depicting inhibition of viral growth using dsRNA compounds of 900 bp wherein 800 bp of GFP is found on either the 3′ or 5′ end of the molecule and the remaining 100 bp is a mismatched plasmid sequence. Both THF (FIG. 8A) and SNB75 (FIG. 8B) cells were exposed to both orientations of this dsRNA compound for 2 h and then exposed to VSV-GFP (MOI=0.1) for 24 h before the supernatant was collected for TCID₅₀ quantification (n=6 for both cell lines). Significant knockdown was observed in both cell lines when GFP was on the 3′ end of the dsRNA compound. When GFP was on the 5′ end, significant inhibition of VSV-GFP was only observed in the THF cells.

FIGS. 9A and 9B are a series of graphs depicting sequence matched dsRNA decreases inducible luminescence in fish cells. RTG-P1 rainbow trout gonadal cells stably express luciferase under the control of an ISRE (interferon response element) thus the presence of IFN activates luciferase production. (FIG. 9A) dsRNA (700 bp) encoding for luciferase (Luc) (match) or GFP (mismatch) were used to treat RTG-P1 cells at varying concentrations (1-25 ng/mL) for 24 h. 1 ng/mL of 700 bp dsRNA, both encoding luciferase or GFP, did not induce IFN. (FIG. 9B) RTG-P1 cells were treated with dsRNA (either Luc (match) or GFP (mismatch) at 1 ng/mL, 10 ng/mL and 25 ng/mL for 4 h prior to treatment with 32 ng/mL poly IC for 24 h. Doses of 1 ng/mL and 10 ng/mL demonstrate a sequence dependent knockdown of luciferase, while 25 ng/mL shows a trend, but is not statistically significant. This suggests that the dsRNAi effect decreases as IFN production increases. Doses 100 ng/mL of dsRNA and above, where IFN production was substantial, demonstrated little to no knockdown in a sequence specific manner (data not shown).

FIG. 10 is a graph showing matched dsRNA sequence decreased CSV viral titre in RTG-2. RTG-2, rainbow trout gonadal cells, were pretreated for 4 hours with 1 ng/mL of GFP (mis-matched), or CSV segment 7 (matched) or CSV segment 10 (matched) sequence specific double stranded RNA, all 700 bp in length, in serum free media. Monolayers were subsequently infected with CSV at an MOI of 1 for 4 hours then replaced with 2% FBS media. Cell supernatant was collected at day 7 post-infection and titred by TCID₅₀ assay. Data (mean+SEM) represented an average of four independent replicates and were analyzed statistically by a student's t-test, alpha=0.05. P value <0.05 considered significant: p 0.05 (*). P>0.05 considered nonsignificant (ns).

FIG. 11 is an image depicting that dsRNAi effect is through the RNAi pathway. Human Embryonic Lung cells (HEL-299; ATCC CCL-137) were pretreated for 2 hours with 500 ng/ml of either mCherry (mis-matched), or eGFP (matched) sequence specific double-stranded RNA in serum free media in the presence or absence of 12.5 μM aurintricarboxylic acid (DICER inhibitor). Monolayers were subsequently infected with VSV-GFP (Indiana) at an MOI of 0.1. Plates were scanned via fluorescence microscopy. Consistent with previous data, the eGFP dsRNA provided superior protection to mCherry dsRNA (compare third column to fifth column). Presence of the DICER inhibitor negated the protective effects (compare third and fourth columns).

FIG. 12A is a schematic of the structure of 700 bp dsRNA compounds containing multiple matched sequences and FIGS. 12B and 12C are a series of graphs depicting treating THF cells with either 500 ng/mL or 1000 ng/mL, respectively, of 700 bp dsRNA containing multiple matched dsRNA sequences. (THF) cells (50,000 cells/well) were exposed to 500 ng/mL (FIG. 12B) or 1000 ng/mL (FIG. 12C) of 700 bp dsRNA containing multiple matched sequences for 2 hours and were then infected with VSV-GFP (multiplicity of infection (MOI)=0.1). Following 24 hours of infection, supernatants were collected and TCID₅₀ was calculated using HEL-299 cells. The dsRNA compounds were designed with either 350 bp VSV N gene sequence and 350 bp VSV M gene sequence together in a 5′ to 3′ direction (5′N-M-3′) or 350 bp VSV M gene sequence and 350 bp VSV N gene sequence together in a 5′ to 3′ direction (5′-M-N-3′) or 50 bp fragments of N and M alternating for 700 bp total (N-M Alt). In the N-M Alt sequence, each fragment was a different part of the N and M gene, ie. no sequences repeated along the dsRNA compound (FIG. 12A). It was found that the 700 bp dsRNA containing multiple matched sequences effectively inhibited viral growth.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

As used herein, “double stranded RNA compound” or “dsRNA compound” refers to a duplex structure comprising two complementary nucleic acid strands, the guide strand and the passenger strand. The guide strand comprises a segment complementary to a target nucleic acid sequence. The dsRNA compound may also comprise one or more nucleotide overhangs.

“Target nucleic acid sequence” as used refers to a portion or all of the target gene transcript, to which the guide strand is complementary. The target nucleic acid sequence is at least 20 bp.

“Target gene transcript” as used herein refers to a mRNA molecule formed during the transcription of a target gene that is desired to be silenced.

As used herein, “complementary” or “complementarity” means the ability of the guide strand to hybridize to the passenger strand or the ability of the segment of the guide strand to hybridize to the target nucleic acid sequence. Complementarity between the segment and the target nucleic acid sequence may be perfect (100% complementary) but some mismatches are tolerated. For example, the segment can be 70%, 80%, 85%, 90% or 95% complementary to the target nucleic acid sequence or comprise up to 1, 2 or 3 mismatches in any 10 monomer stretch.

As used herein, “guide strand” refers to a single-stranded nucleic acid molecule of a double-stranded nucleic acid molecule or a double stranded nucleic acid-containing molecule that has a segment that is sufficiently complementary to a target nucleic acid sequence to cause RNA interference.

As used herein, a “passenger strand” refers to a single-stranded nucleic acid molecule of a double-stranded nucleic acid molecule or a double stranded nucleic acid-containing molecule that has a sequence that is complementary to the segment of the guide strand.

As used here, “blunt end” refers to a terminus of a dsRNA compound as having no overhanging nucleotides. The dsRNA compound herein described can for example comprise blunt ends at one or both termini of the duplex structure.

As used herein, “overhang” refers to unpaired nucleotides, in the context of a duplex having two, three or four free ends at either the 5′ terminus or 3′ terminus of a double-stranded nucleic acid. The overhang can be for example, a 3′ or 5′ overhang on the guide strand or passenger strand or both and can be native or non-native.

As used herein, “non-native” in the context of the dsRNA compound, including the guide and passenger strands and overhang sequences, refers to a nucleotide sequence or residues that are not comprised in or correspond to (e.g. complementary to) the target nucleic acid sequence or target gene transcript.

As used herein, “chemical modification” can be one found for example in locked nucleic acids (LNAs) or can be 2′-fluoro (2′-F), 2′-O-methoxyethyl (2′-MOE) or 2′-O-methyl (2′-O-Me), which are modifications at the 2′ position of the ribose moiety or morpholino monomer where a six-membered morpholine ring replaces the sugar moiety or phosphorothioate (PS) linkage where sulfur replaces one of the non-bridging oxygen atoms in the phosphate group. Such modifications may increase stability in the presence of nucleases.

As used herein, “conjugate” means a compound comprising two or more molecules that are covalently linked, for example an antibody or label linked, for example via a linker, to a dsRNA compound herein disclosed.

As used herein, “interferon induction threshold” or “IFN induction threshold” refers to the concentration of a dsRNA compound and/or the length of a dsRNA compound which upon administration to an interferon-competent cell or subject, will trigger an interferon response in the cell.

As used herein, “interferon-competent cell” or “IFN-competent cell” refers to a cell that is capable of producing type I IFNs.

As used herein, “interferon stimulated genes” refers to genes whose expression is stimulated by type I interferons and includes for example, ISG15, CXCL10 etc.

The term “treating” in the context of a subject means an approach for obtaining beneficial or desired results, including clinical results. Beneficial or desired clinical results can include, but are not limited to, decreasing of infectivity, inhibiting viral growth, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject susceptible to contracting an infection, e.g. fish in a farm lake or pond where infection is present, or during infection season, can be treated prophylactically by administering a dsRNA compound, vector, conjugate or composition herein described to prevent or reduce pathogen infectivity. The term “treating” in the context of a cell, means administering to or contacting the cell, such as by soaking the cells in a solution. Cells can be treated without the need for a transfection reagent as demonstrated herein.

As used herein, the term “administration” means to provide or give a cell or a subject an agent, such as a dsRNA compound, vector, conjugate and/or composition described herein, using an effective route. For example, the dsRNA compound may be administered to a subject intranasally (e.g. aerosol spray), topically, orally, rectally, vaginally, by bath and/or intravenously etc. The administration may also be carried out by treating the subject (e.g. fish) or cell by placing the cell or subject in a solution containing the dsRNA compound, vector, conjugate and/or composition suspended for example in a medium or adding the solution containing the dsRNA compound, vector, conjugate and/or composition into the existing milieu of the cell or subject (e.g. into cell medium or liquid housing the fish or other aquatic vertebrate).

The term “pharmaceutically acceptable carrier” as used herein means a carrier suitable for administration of an agent, such as a dsRNA compound, vector, and/or composition described herein, and includes, without limitation, saline, buffered saline, dextrose, water, glycerol, ethanol, and combinations thereof.

The term “silencing” as used herein means decreasing the level of mRNA expression of a target gene, by for example at least 10%, at least 20%, at least 30%, at least 40%, 50%, at least 60%, at least 70%, at least 80%, at least 90%, at least 95% or more detectable for example by RT-PCR using for example conditions described herein. The RT-PCR conditions can for example include 35 cycles and be compared the level of cDNA amplified in a control not treated with the dsRNA compound described herein.

The term “antibody” as used herein is intended to include human antibodies, monoclonal antibodies, polyclonal antibodies, single chain and other chimeric antibodies. The antibody may be from recombinant sources and/or produced in transgenic animals. The antibody in an embodiment comprises a heavy chain variable region or a heavy chain comprising a heavy chain complementarity determining region 1, heavy chain complementarity determining region 2 and heavy chain complementarity determining region 3, as well as a light chain variable region or light chain comprising a light chain complementarity determining region 1, light chain complementarity determining region 2 and light chain complementarity determining region 3. As used herein, “antibody” also refers to antibody binding fragments, including, which includes, without limitation, Fab, Fab′, F(ab′)2, scFv, scFab, dsFv, ds-scFv, dimers (e.g. Fc dimers), minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, scFab, dsFv, ds-scFv, dimers, minibodies, diabodies, bispecific antibody fragments and other fragments can also be synthesized by recombinant techniques.

As used herein, “subject” includes all vertebrate animals including vertebrate mammals and non-mammals, for example, humans, fish and other aquatic vertebrates, cows, pigs, horses, birds (e.g. poultry), amphibians, reptiles, etc.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous. Any feature that is recited in a list may be combined with any other feature that is recited in another list.

The inventors have developed compositions and methods for decreasing expression of a target gene or genes (i.e. silencing expression) in a vertebrate cell or vertebrate subject using long double stranded RNA without inducing type I IFN, thereby interrupting the negative feedback loop preventing RNAi. The compositions and methods are robust and may be used for effectively targeting multiple gene transcripts or multiple segments in a gene transcript ensuring efficient knock down.

Accordingly, a first aspect includes a double stranded RNA (dsRNA) compound (e.g. non-naturally occurring recombinant construct) comprising a guide strand and a passenger strand, the guide strand and the passenger strand each having a length of at least 300 basepairs (bp), and the guide strand comprising a segment complementary to a target nucleic acid sequence of a target gene transcript.

In an embodiment, the guide strand or a segment thereof may be about 70%, about 80%, or about 90% complementary to the target nucleic acid sequence. The guide strand or segment thereof may be perfectly complementary (100% complementary) to the target nucleic acid sequence. In an embodiment, the guide strand and/or the passenger strand are chimeric e.g. contain two or more chemically distinct regions. For example, the chimeric strand may contain one or more regions of modified nucleotides that confer beneficial properties (e.g., increased nuclease resistance, increased uptake into cells).

As compared to other dsRNA molecules which only induce an IFN response, the complementarity of the guide strand to the target nucleic acid sequence allows the dsRNA compound to facilitate degradation of a target mRNA sequence or inhibition of translation of the mRNA target sequence.

The target nucleic acid sequence can for example be a host gene transcript sequence (i.e. portion of a host gene transcript) or a pathogen or non-host gene transcript sequence. A single dsRNA compound may contain in its guide strand multiple segments each complementary to different target nucleic acid sequences for example in succession along a particular target gene transcript, or found in different gene transcripts enabling a single dsRNA compound to target multiple gene transcripts via RNAi. In an embodiment, the guide strand comprises one or more further segments each complementary to a different target nucleic acid sequence of the same target gene transcript. In another embodiment, the one or more further different segments are complementary to different target nucleic acid sequences of different target gene transcripts, and are optionally are separated by a buffer sequence (or mismatched sequence), for example a sequence of about 50 bp to about 500 bp, or longer, that is not complementary to a target nucleic acid sequence. In an embodiment, the guide stand comprises one or more further segments each complementary to the target nucleic acid sequence. For example, the dsRNA compound may contain alternating target nucleic acid sequences from two different target gene transcripts. The length of each segment complementary to a target nucleic acid sequence can be for example, about 20 bp to about 800 bp, or longer. The length of the whole dsRNA compound can be about 300 bp to about 5000 bp, or longer, optionally about 3000 bp.

In some embodiments, the segment that is complementary to the target nucleic acid sequence has a length of between 20 bp and 850 bp, for example about 20 bp, about 25 bp, about 40 bp, about 50 bp, about 60 bp, about 75 bp, about 80 bp, about 100 bp, about 200 bp, about 300 bp, about 300 bp, about 400 bp, about 500 bp, about 600 bp, about 700 bp or about 800 bp, or from 20 to 800 bp, 40 to 700 bp, 60 to 600 bp, 80 to 500 bp, 100 to 500 bp, 200 to 500 bp, 300 to 500 bp or 400 to 500 bp.

In an embodiment, the segment of the guide strand that is complementary to the target nucleic acid sequence is proximal to (e.g. less than 50 bases) or at the 5′ end of the guide strand. “Proximal to the 5′ end” as used herein means closer, e.g. at least 3 nucleotides closer, to the 5′ end than the 3′ end. In another embodiment, the segment is proximal to or at the 3′ end of the guide strand. “Proximal to the 3′ end” as used herein means closer, e.g. at least 3 nucleotides closer, to the 3′ end than the 5′ end. In embodiments where the dsRNA compound comprises multiple segments, one segment may be proximal to or at the 5′ end and another segment may be proximal to or at the 3′ end. Other segments comprised in the dsRNA compound are located between the segments proximal to or at the ends of the guide strand, optionally separated by a buffer sequence.

In an embodiment, the guide strand comprises two different segments each complementary to different target nucleic acid sequences of the same target gene transcript, the first segment being proximal to or at the 3′ end of the guide strand and the second segment being proximal to or at the 5′ of the guide strand. In another embodiment, the guide strand comprises two different segments each complementary to a target nucleic acid sequence of a different target gene transcript, the first segment being proximal to or at the 3′ end of the guide strand and the second segment being proximal to or at the 5′ of the guide strand.

In some embodiments, the guide strand comprises one or more non-native sequences. For example, the non-native sequence can be a non-native RNA sequence, for example a buffer sequence.

The dsRNA compound comprises two single stranded (ss) RNA molecules of complementary sequence—the passenger strand and the guide strand—annealed together.

The dsRNA compound may be synthesized by any desired procedure. Various procedures are well-known in the art including, for example, in vitro transcription using RNA polymerase (e.g. T7 RNA polymerase), bacteria, large scale fermentation processes, and the like. In one embodiment, the dsRNA compound may be synthesized in guide and passenger strands from a DNA template containing polymerase promoters, and complementary sequences made by the polymerase, which anneal to make the dsRNA compound. In an embodiment, the polymerase promoter is a T7, T3 or SP6 polymerase promoter. In an embodiment, the polymerase promoter is a T7 polymerase promoter.

In some embodiments, the guide strand and the passenger strand each have a length of between 300 bp and 5000 bp, for example about 300 bp, about 400 bp, about 500 bp, about 600 bp, about 700 bp or about 800 bp, 900 bp, 1000 bp, 1100 bp, about 1200 bp, about 1300 bp, about 1400 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp or from 300 to 900 bp, 400 to 900 bp, 500 to 900 bp, 600 to 900 bp, 700 to 900 bp, 300 to 800 bp, 400 to 800 bp, 500 to 800 bp, 600 to 800 bp, 700 to 800 bp, 300 to 3000 bp, 400 to 3000 bp, 300 to 2000 bp or from 400 to 2000 bp. The minimum length can be any whole number between 300 and 400 bp. The length can be any whole number between and including 300 to 3000 bp. Longer sequences may also be used.

In an embodiment, the guide strand and the passenger strand each have a length of at least 300 bp. In an embodiment, the guide strand and the passenger strand each have a length of at least of at least 400 bp. In an embodiment, the guide strand and the passenger strand each have a length of at least 500 bp. In an embodiment, the guide strand and the passenger strand each have a length of at least 600 bp. In an embodiment, the guide strand and the passenger strand each have a length of at least 700 bp. In an embodiment, the guide strand and the passenger strand each have a length of at least 800 bp. In an embodiment, the guide strand and the passenger strand each have a length of at least 900 bp. In an embodiment, the guide strand and the passenger strand each have a length of at least 1000 bp. In an embodiment, the guide strand and the passenger strand each have a length of up to 2000 bp. In an embodiment, the guide strand and the passenger strand each have a length of up to 1500 bp. In an embodiment, the guide strand and the passenger strand each have a length of up to 2000 bp. In an embodiment, the guide strand and the passenger strand each have a length of up to 1500 bp. In an embodiment, the guide strand and the passenger strand each have a length of up to 1300 bp. In an embodiment, the guide strand and the passenger strand each have a length of up to 1200 bp. In an embodiment, the guide strand and the passenger strand each have a length of up to 1100 bp.

In an embodiment, the dsRNA compound comprises at least one blunt end, optionally two blunt ends. In some embodiments, the guide strand and/or the passenger strand comprises an overhang. In one embodiment, the overhang is complementary to the target nucleic acid sequence. In another embodiment, the overhang is non-native.

In one embodiment, the dsRNA compound comprises one or more chemically modified bases. In one embodiment, the chemical modification is selected from 2′Omethyl (2′-O-Me), 2′-O-methoxyethyl (2′O-MOE), 2′fluoro (2′F), and locked nucleic acid monomer (LNAM). In another embodiment, the dsRNA compound is chimeric.

In another embodiment, the dsRNA compound comprises non-modified RNA.

In an embodiment, the guide strand and/or the passenger strand comprises a 5′-triphosphate end. In an embodiment, the guide strand and/or the passenger strand comprises a 5′-diphosphate end. In an embodiment, the guide strand and/or the passenger strand comprises a 5′-monophosphate end. In an embodiment, the guide strand and/or the passenger strand lacks a 5′-triphosphate end.

In an embodiment, the dsRNA compound does not comprise a replication gene, optionally a viral replication gene.

In some embodiments, the target gene transcript is a pathogen gene transcript such as a viral gene transcript. Viral genes of particular interest include genes coding for structural proteins, non-structural proteins, immune-evasion proteins, and polymerases. The viral gene can be from any virus including but not limited to influenza virus, coronavirus, rhabdoviruses, rabies, ebolavirus, dengue virus, rotavirus. In another embodiment, the viral gene is a Vesicular stomatitis Indiana virus (VSV) gene. In a further embodiment, the viral gene transcript is a membrane (M) protein gene transcript. In a further embodiment, the viral gene is a nucleocapside (N) protein gene transcript. In a further embodiment, the viral gene is the spike (S) protein gene transcript. In an embodiment, the viral gene transcript is influenza virus, coronavirus, rabies, ebolavirus, dengue virus, rotavirus or rhabdovirus transcript. For example, the coronavirus transcript is human coronavirus transcript, such as human coronavirus SARS-CoV-1, SARS-CoV-2, MERSr-CoV, HCoV-229E, HCoV-OC43, HCoV-NL63 and HCoV-HKU1 transcripts.

The target gene transcript for RNAi silencing is not particularly limited. The nucleic acid (e.g. gene transcript) to be targeted depends on the type of therapy envisaged. In some embodiments, the target gene transcript is a disease gene transcript, optionally an oncogene transcript. In one embodiment, the disease is cancer. In another embodiment, the disease is a lymphoproliferative disorder such as plasma cell proliferative disorder.

Some non-limiting examples of target gene transcripts include target gene transcripts disclosed in U.S. Pat. No. 8,697,359 issued Apr. 15, 2014, the contents of which are herein incorporated by reference. Specific examples of target gene transcripts include, for example, transcripts of developmental genes (e.g., adhesion molecules, cyclin kinase inhibitors, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM1, PML, RET, SRC, TALI, TCL3, andYES); tumor suppressor genes (e.g., APC, BRCA1, BRCA2, MADH4, MCC, NF1, NF2, RB 1, TP53, and WTI); genes that encode enzymes (e.g., ACC Synthasesandoxidases, ACP desaturases and hydroxylases, ADP-glucosepyrophorylases, ATPases, alcohol dehydrogenases, amylases, amyloglucosidases, catalases, cellulases, chalcone synthases, chitinases, cyclooxy genases, decarboxylases, dextrinases, DNA and RNA polymerases, galactosidases, glucanases, glucoseoxidases, granule-bound starch synthases, GTPases, helicases, hemicellulases, integrases, inulinases, invertases, isomerases, kinases, lactases, upases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pulanases, recombinases, reverse transcriptases, RUBISCOS, topoisomerases, and xylanases); and, genes that encode chemokines (e.g.CXCR4, CCR5), the RNA component of telomerase, vascular endothelial growth factor (VEGF), VEGF receptor, tumor necrosis factors nuclear factor kappa B, transcription factors, cell adhesion molecules, insulin-like growth factor, transforming growth factor beta family members, cell surface receptors, RNA binding proteins (e.g. small nucleolar RNAs, RNA transport factors), translation factors, telomerase reverse transcriptase), etc.

Another aspect is a conjugate comprising the dsRNA compound described herein and one or more polypeptide, such as an antibody, optionally a single chain antibody or a binding fragment, or a label.

In an embodiment, the conjugate comprises the dsRNA compound and an antibody.

In an embodiment, the conjugate comprises the dsRNA compound and a label such as a detectable label.

Nucleic acids may be modified with a detectable label to enable detection or purification. Examples of labels include radioactive phosphates, biotin, fluorophores and enzymes. Nucleic acids can also be part of a conjugate to facilitate for example targeted delivery or immobilization. For example, nucleic acid molecules can be labeled with tags or other modifications during synthesis. Nucleic acid molecules can also be labelled post synthesis. Enzymatic and chemical methods are available as well as click chemistry reagents.

Antibodies or peptides can be conjugated to dsRNA compounds described herein using various methods. For example, EDC (also called EDAC) which is 1-ethyl-3-(-3-dimethylaminopropyl) carbodiimide hydrochloride, can be used. Nucleic acid molecules containing a 5′-phosphate group can be reacted with EDC optionally in combination with imidazole, to create nucleotide phosphoramidate conjugates. Antibodies can also be conjugated using biotin avidin based click chemistry conjugation. Sodium meta-periodate can be used as the oxidizer of protein carbohydrates to generate reactive aldehyde groups for chemical conjugation procedures. The vicinal diols of ribose in RNA nucleotides can also be cleaved with periodate, enabling this method to be used to add a single 3′-end protein to RNA. Other methods for conjugating the dsRNA compounds herein described include using dual variable domain antibodies containing a reactive lysine residue suitable for site-specific conjugation to beta-lactam linker-functionalized siRNA, as described in Nanna, A R et al. 2020 (Nucleic Acids Research, Volume 48, Issue 10, 4 Jun. 2020, pages 5281-5293). The dsRNA compound can also be used as a linker that non-covalently connects antibodies to other peptides such as drugs compounds, forming antibody-drug conjugates, as described in Dovgan I et al. 2020 (Sci Rep 10, 7691).

Another aspect includes a vector comprising an expression cassette encoding one or more transcript(s) that can form the dsRNA compound described herein (e.g. self-anneal). In some embodiments, the expression cassette comprises RNA polymerase promoters e.g. T7 polymerase promoters, for producing the guide strand and the passenger strand. In another embodiment, the expression cassette comprises an RNA polymerase promoter, e.g. T7 polymerase promoter, for a transcript that can self-anneal to form the dsRNA compound. In another embodiment, the vector is a plasmid, viral construct or virus. In a further embodiment, the virus is a lentivirus or an adeno associated virus. In an embodiment where the vector is for use in vivo, a strong expression promoter e.g. cytomegalovirus (CMV) promoter may be used.

Another aspect includes a composition comprising the dsRNA compound, the conjugate, or the vector described herein.

In another embodiment, the composition further comprises a pharmaceutically acceptable carrier.

In another embodiment, the composition further comprises lipid transport particles. In a further embodiment, the lipid transport particles are liposomes. In a further embodiment, the lipid transport particles are nanoparticles. In a further embodiment, the lipid transport particles are nanosomes.

In an embodiment, the composition comprises cell culture media, optionally serum free cell culture media.

In an embodiment, the composition lacks a delivery mechanism such as a lipid transport particles.

In another embodiment the concentration (e.g. weight per volume) of the dsRNA compound in the composition is in a dosage amount that is below a selected IFN induction threshold. The IFN induction threshold can be selected according to the use or route of administration and can be based on the average, median or mean threshold of a group of cells. The concentration below IFN induction threshold may be dependent on cell and subject type as well as route of administration. In one embodiment, the concentration is up to 5000 ng/mL. In one embodiment, the concentration is up to 1500 ng/mL. In another embodiment, the concentration is between 1 ng/mL to 5000 ng/mL. In another embodiment, the concentration is 1 ng/mL, 2 ng/mL, 3 ng/mL, 4 ng/mL, 5 ng/mL, 10 ng/mL, 15 ng/mL, 20 ng/mL, 50 ng/mL, 100 ng/mL, 200 ng/mL, 300 ng/mL, 400 ng/mL, 500 ng/mL, 600 ng/mL, 750 ng/mL, 1000 ng/mL, 1250 ng/mL, 1500 ng/mL, 1750 ng/mL, 2000 ng/mL, 2250 ng/mL, 2500 ng/mL, 2750 ng/mL, 3000 ng/mL, 3500 ng/mL, 4000 ng/mL, 4500 ng/mL or 5000 ng/mL. In another embodiment, the concentration is 500 ng/mL, 1000 ng/mL, or 1500 ng/mL. In an embodiment, the number of dsRNA molecules per cell when 100 ng/mL dsRNA is delivered to the cell is about 55, 800 dsRNA molecules per cell.

Various uses can be made of the dsRNA compounds, vectors, conjugates and compositions. For example, the foregoing can be used for anti-pathogen therapy where the long dsRNA compound encoding for pathogen sequences induces (1) IFN-associated pathways to shut down pathogen replication and induce recruitment and activation of innate and adaptive immune cells at the site of infection and (2) RNAi to shut down expression of pathogen genes required for replication. A dsRNA compound, vector, conjugate or composition administered to a cell at a certain concentration may induce IFN, while administering the same dsRNA compound, vector, conjugate or composition to the same cell at a different e.g. lower concentration may induce RNAi. Administering the same dsRNA compound in the same concentrations to two different cell types may induce IFN in one cell type, while inducing RNAi in the other cell type. This therapy could be used for both prophylactic and infection treatment regimens.

Another example is anti-cancer therapy where the long dsRNA compound can be designed to target one or more oncogene transcript and induce (1) IFN associated pathways in IFN-competent tumor cells and cells surrounding the tumor cells to recruit and activate innate and adaptive immune cells within the tumor and surrounding the tumor and (2) RNAi to shut down oncogene expression in IFN-incompetent tumor cells.

Also the compounds, vectors, conjugates and compositions described herein can be used in methods to provide a tunable interferon regulatory mechanism for example by identifying the IFN induction threshold and using the induction threshold to select the concentration of dsRNA compound to administer based on desired response, IFN or RNAi. This application could be combined with anti-pathogen or anti-cancer therapy for example to push the response towards IFN or RNAi as needed.

Accordingly, another aspect includes the use of the dsRNA compound, vector, conjugate and composition herein described in silencing a target gene transcript in a vertebrate cell or subject comprising the target nucleic acid sequence, reducing replication or infectivity of a pathogen infection in the vertebrate cell or subject.

In another embodiment, the dsRNA compound is delivered into the cell using a bacteriophage. In another embodiment, the dsRNA compound is delivered into the cell using a vector, optionally a plasmid, viral construct or virus vector. In another embodiment, the dsRNA compound is delivered into the cell using a nanoparticle, peptide, liposome, or virus-like particle.

Another aspect is a use of the dsRNA compound, vector, conjugate or composition herein described for reducing replication or infectivity of a pathogen infection in the vertebrate cell or subject.

Another aspect is a use of the dsRNA compound, vector, conjugate or composition herein described for treating a pathogen infection in a subject in need thereof.

Another aspect is a use of the dsRNA compound, vector, conjugate or composition herein described for the preparation of a medicament.

In an embodiment, the medicament is for treatment of pathogen infection such as viral infection. In an embodiment, the medicament is for treatment of cancer.

In an embodiment, the medicament is for treatment of a disease relating to or involving a target gene transcript, herein described, targeted for RNAi silencing. As mentioned, the target gene transcript for RNAi silencing is not particularly limited. The gene transcript to be targeted depends on the type of therapy envisaged. In some embodiments, the target gene transcript is a disease gene transcript, optionally an oncogene transcript. In one embodiment, the disease is cancer. In another embodiment, the disease is a lymphoproliferative disorder such as plasma cell proliferative disorder.

Another aspect includes a method of silencing a target gene transcript in a vertebrate cell or subject, the method comprising administering to the cell or subject an amount of the dsRNA compound, vector, conjugate, or composition described herein. Vertebrates include mammals and non-mammals, for example, humans, cows, pigs, horses, fish, birds (e.g. poultry), amphibians, reptiles, etc.

The method is particularly useful for treating cells undergoing insult by a pathogen, for example viruses, bacteria, fungi, protozoans or other parasites (for example, multicellular parasites (e.g. worms)).

Cell types include, for example, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. Of particular note, are stem cells, cancer cells, tumor cells, cells in the lung, cells of the skin, cells of the intervertebral disc, cells of the eye, cells of the brain and the like.

The method may involve the use of dsRNA compounds that contain a plurality of different segments, each of the different segments silencing a different target gene transcript. In this manner, a single dsRNA compound can be used to target different gene transcripts for more effective therapeutic effect and/or to personalize treatment to an individual patient's needs.

In an embodiment, the amount of the dsRNA, vector, conjugate, or composition administered is below a predetermined IFN induction threshold. In an embodiment, the amount of the dsRNA, vector, conjugate, or composition administered is above a predetermined IFN induction threshold.

The predetermined IFN induction threshold can be determined based on a comparator cell, subject, population of cells or population of subjects. In an embodiment, the predetermined IFN induction threshold is determined using a comparator population of cells, wherein the comparator population of cells is incubated with the dsRNA compound, vector, conjugate or composition and the predetermined IFN induction threshold is determined therefrom. In an embodiment, the cells are incubated with the dsRNA compound, vector, conjugate or composition, for about 2 hours. Other times can also be used. The IFN induction threshold can be determined depending on the intended use. For example the induction threshold can be determined by incubating the dsRNA compound with the cell at infection, after infection or prior to infection. In another embodiment, the cells are incubated with the dsRNA compound, vector, conjugate or composition up to about 48 hours prior to infection. In another embodiment, the cells are incubated with the dsRNA compound, vector, conjugate or composition up to about 24 hours prior to infection. In a further embodiment, the cells are incubated with the dsRNA compound, vector, or composition about 1 hour to 5 hours prior to infection, optionally, 2 hours, 3 hours, of 4 hours prior to infection. In another embodiment, the cells are incubated with the dsRNA compound, vector, conjugate or composition at the time of infection.

In embodiments where the method is for treating a subject, the predetermined IFN induction threshold can be obtaining by administering to a comparator population of subjects (e.g. having a particular infection or cancer) different concentrations and/or lengths of the dsRNA compound to determine the dosages and/or lengths that fail to induce and which induce an IFN response. The determining the IFN induction threshold in the population of subjects can for example be preceded by pre-clinical assessment for example, pre-clinical animal trials, to determine dosages that do not induce IFN but that do induce RNAi.

In another embodiment, the method of determining the predetermined IFN induction threshold comprises incubating the comparator population of cells with or administering to the comparator population of subjects a dsRNA compound, or vector or composition comprising a dsRNA compound between 300 bp and 900 bp, for example about 300 bp, about 400 bp, about 500 bp, about 600 bp, about 700 bp or about 800 bp, or from 300 to 900 bp, 400 to 900 bp, 300 to 800 bp or from 400 to 800 bp and identifying if an IFN response occurs in the comparator population of cells or subjects.

In another embodiment, the method for determining the predetermined IFN induction threshold comprises delivering to the comparator population of cells or administering to the comparator population of subjects different concentrations and/or lengths of dsRNA compound to determine the dosages and/or lengths of dsRNA compound that fail to induce and which induce an IFN response. The comparator population can be a cell line, primary cell culture, primary tissue culture or animal exposure, or a group of subjects having or at risk of having a disease or pathogen infection.

Dosages may be determined using one or more delivery methods e.g. the intended delivery method to be used. In one embodiment, the different concentrations and/or lengths of dsRNA compound, vector, or composition is delivered via incubating the comparator population in the dsRNA compound, vector, or composition. In other embodiments, the delivery method may include a bacteriophage, vector, optionally a plasmid, viral construct, or virus, nanoparticle, peptide, liposome, or virus-like particle comprising or able of expressing the dsRNA compound.

The comparator population of cells is typically the same cell type as the vertebrate cell e.g. the cell to be treated with the dsRNA compound. The comparator population or subjects has or is at risk of having the same disease or infection as the subject to be treated with the dsRNA compound.

Different cell types can be treated with the compounds described herein simultaneously. Each cell type may have a different IFN induction threshold which can be exploited. For example the first cell can have a first threshold concentration for the dsRNA compound and the second cell can have a second threshold concentration for the dsRNA compound, where the first threshold concentration is lower than the second threshold concentration, for example in a tumor microenvironment or any tissue that is of a mixed cell type. The concentration of the dsRNA compound can be titrated to be above the first threshold and below the second threshold where for example it is desirable to induce an IFN response in the first cell and gene transcript silencing in the second cell, for example in tumor microenvironment.

In another embodiment, identifying IFN induction threshold in the comparator population of cells or of subjects comprises measuring the expression levels of interferon-stimulated genes (ISG). In an embodiment, the expression levels of ISGs are measured using qPCR. In another embodiment, the expression level of ISGs is measured using RT-PCR, RNAseq, and/or fluorescence in situ hybridization (FISH). In another embodiment, the expression levels of ISGs are measured about 24 hours after incubation or treatment with the dsRNA compound.

In an embodiment the cell is a vertebrate cell is a disease cell, such as a cancer cell.

Another aspect includes a method of targeting a mixed cell population by silencing a target gene transcript in a first vertebrate cell type comprising the target nucleic acid sequence and inducing a type I interferon (IFN) response in a second vertebrate cell type, optionally wherein the cell population is in a vertebrate subject

Another aspect includes a method of reducing replication or infectivity of a pathogen infection in the vertebrate cell or subject, respectively, or for treating a pathogen infection in a subject in need thereof wherein the target nucleic acid sequence corresponds to a pathogen gene transcript sequence, such as a viral gene transcript sequence.

In one embodiment, the administering is prior to exposure to the pathogen. In another embodiment, the administering is during exposure to the pathogen.

Another aspect includes a method of treating a pathogen infection the method comprising administering to a subject in need thereof an amount of the dsRNA compound, vector, conjugate or composition described herein, wherein the target gene transcript is a pathogen gene transcript, such as a viral gene transcript. In one embodiment, the pathogen gene(s) may be one or more human coronavirus gene(s), optionally from a human coronavirus, such as human coronaviruses SARS-CoV-1, SARS-CoV-2, MERSr-CoV, HCoV-229E, HCoV-OC43, HCoV-NL63 and HCoV-HKU1. In a further embodiment, the one or more coronavirus gene(s) may be the gene(s) encoding the N protein, M protein and/or Spike protein.

The dsRNA compound, vector, conjugate or composition can for example, be administered prior to infection, at infection, and/or after infection. In an embodiment, the dsRNA compound, vector, conjugate or composition can be used as a prophylactic treatment, for example, where a family member is identified as infected and family members take it as a prophylactic before showing signs.

In another embodiment, the pathogen infection has a site of infection and the amount is administered to the site of infection (e.g. locally). In some embodiment, the amount is administered intranasally, orally, topically, ocularly, rectally, vaginally, by bath, subcutaneously, intravenously, intraperitoneally, intrapleurally or intramuscularly. In an embodiment, the dsRNA compound, vector, conjugate or composition is administered intranasally and is in the form of an aerosol. In another embodiment, the dsRNA compound, vector, conjugate or composition is administered topically and is in the form of a cream, gel, ointment, paste, colloid or suspension.

In another embodiment, the amount administered to the site of infection is above an IFN induction threshold.

In another embodiment, the amount is administered systemically and the amount is below an IFN induction threshold. In some embodiments, the administration is given topically and/or through intravenous injection and/or intratumorally.

In another embodiment, the cell is an aquatic vertebrate cell. In an embodiment, the subject is an aquatic vertebrate.

In another embodiment, the cell is a mammalian cell.

In another embodiment, the cell is a cancer cell or a cell from an immune-privileged site. An “immune-privileged site” means a site that site that is able to tolerate the introduction of foreign substances without rejection, for example, the eyes, placenta, fetus, testes, central nervous system, and/or hair follicles etc.

In another embodiment, the subject is an aquatic vertebrate. In an embodiment, the aquatic vertebrate is a fish such as a teleost.

In another embodiment, the subject is a terrestrial vertebrate, in an embodiment the terrestrial vertebrate is a bird.

In a further embodiment, the subject is a mammal, optionally a human. Other mammals include any agricultural species such as avian, bovine, porcine or ovine species. Domestic animals are also contemplated. In another embodiment, the mammal is a cow, pig, horse or sheep.

In an embodiment, the dsRNA compound, vector, conjugate or composition is not administered in the presence of a delivery mechanism such as a transfection reagent.

Another aspect is a use of the dsRNA compound, vector, conjugate or composition herein described for silencing a target gene transcript in a vertebrate cell or subject.

Another aspect is a use of the dsRNA compound, vector, conjugate or composition herein described for reducing replication or infectivity of a pathogen infection in the vertebrate cell or subject.

Another aspect is a use of the dsRNA compound, vector, conjugate or composition herein described for treating a pathogen infection in a subject in need thereof.

Another aspect is a use of the dsRNA compound, vector, conjugate or composition herein described for the preparation of a medicament. In an embodiment, the medicament is for use in the treatment of a pathogen infection or a disease relating to or involving a target gene transcript, optionally a target gene transcript described herein.

Applications that require induction of IFNs and/or RNAi induced by the dsRNA compound, vector, conjugate or composition herein described that are not listed above would also be appropriate and included in the potential applications for the dsRNA compound, vector, conjugate or composition.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Methods

### Maintenance of Cell Lines

### Mammalian Cell Lines

All procedures using mammalian cells were completed at 37° C. with 5% C02. Routine cell maintenance occurred in vented T75 flasks (BD Falcon).

Human embryonic lung cells (HEL-299) and telomerase-immortalized human fibroblasts (THF) were routinely maintained in Dulbecco's Modified Eagle Media (DMEM, Sigma) supplemented with 10% fetal bovine serum (FBS, Seradigm) and 1% penicillin/streptomycin (Sigma). Unless otherwise specified, all procedures involving these cell lines use this media formulation. Cells were subcultured at a ratio of 1:6 every 2-3 days using 0.25% trypsin (VWR) to dissociate the cells.

Human melanoma cells (M14), human breast carcinoma cells (BT549) and human glioblastoma cells (SNB75) were routinely maintained in Roswell Park Memorial Institute (RPMI)-1640 medium (Corning) supplemented with 10% FBS and 1% penicillin/streptomycin. Unless otherwise specified, all procedures involving these cell lines use this media formulation. Cells were subcultured at a ratio of 1:4 every 2-3 days using 0.25% trypsin to dissociate the cells.

MRC5, human lung fibroblasts, and HepG2, human liver epithelial cells, were both routinely maintained in Eagle's Minimum Essential Medium supplemented with 10% FBS and 1% penicillin/streptomycin. Unless otherwise specified, all procedures involving these cell lines use this media formulation. Cells were subcultured at a ratio of 1:4 every 2-3 days using 0.25% trypsin to dissociate the cells.

### Teleost Cell Lines

RTG-P1 is a rainbow trout gonadal cell line that stably expresses luciferase under the control of an interferon stimulated response element (ISRE), thus the presence of interferon (IFN) activates luciferase production. RTG-2 is the non-genetically altered source cell line for RTG-P1. Both cell lines were routinely maintained in Leibovitz-15 media (L-15, Gibco) supplemented with 10% FBS and 1% penicillin/streptomycin. Unless otherwise specified, all procedures involving these cells use this media formulation. The cells were maintained at 20° C. and routine cell maintenance occurred in plugged T75 flasks (BD Falcon). RTG-P1 and RTG-2 cells were subcultured at a ratio of 1:2 on a bi-weekly basis using 0.25% trypsin to dissociate the cells.

### Virus Propagation

The Indiana serotype of Vesicular Stomatitis Virus (VSV-GFP) contains a GFP gene incorporated between the viral G and L genes. As a result, cells infected with VSV-GFP fluoresce green. VSV-GFP was propagated on monolayers of HEL-299 cells. Virus infections were performed in DMEM containing 10% FBS and 1% penicillin/streptomycin at 37° C. with 5% C02. Virus-containing media was cleared of cellular debris by centrifugation at 4000×g for 4 min followed by filter sterilization through a 0.45 μm filter. The 50% tissue culture infective dose (TCID50/mL) values for the viral stocks were quantified as described below.

### Tissue Culture Infective Dose (TCID50)

Virus-containing media was centrifuged at 4000×g for 4 min to remove cellular debris. VSV-GFP production was measured by TCID50/mL on HEL-299 cells seeded in a 96-well plate (1.5×104 cells/well). 24-36 h post infection wells were scored by fluorescence microscopy (presence of eGFP fluorescence was considered positive infection). Resultant TCID50/mL values were calculated using the Reed and Muench method.

Synthesis of dsRNA Compounds

The dsRNA compound is synthesized in sense and antisense strands from a DNA template containing T7 polymerase promoters, and complementary sequences are made by the T7 polymerase, which anneal to make the dsRNA compound. The dsRNA compound in this technology is not limited to the above method of synthesis, but could include any method of synthesizing long (>100 bp) dsRNA compounds containing a specific sequence.

Genes of interest were amplified using forward and reverse primers that contained T7 promoters. The primer sets and their associated templates are outlined in Table 1. The DNA products were amplified by PCR using 10 ng of appropriate template (Table 1), 2X GOTaq colorless master mix (Promega), 0.5 μM of both forward and reverse primers (Table 1, SigmaAldrich), and nuclease free water to a final volume of 50 μL. The following protocol was carried out in a Bio-Rad T100 thermocycler: 98° C.—5 min, 34 cycles of 98° C.—10 s, 50° C.—10 s, 72° C.—50 s, followed by 72° C.—5 min. The resulting DNA amplicons with T7 promoters on both DNA strands was purified using a QIAquick PCR purification kit (Qiagen). The purified product was then used in the MEGAScript RNAi Kit (Ambion) as per the manufacturer's instructions to produce the dsRNA compound. To confirm primer specificity, 100 ng of all PCR amplicons and the final dsRNA compound were separated on 1% agarose gels containing 1% GelGreen (Biotium Inc.).

TABLE 1
Primers with T7 promoter sequences that were used for amplification
of genes of interest. The resulting DNA amplicons were then used for dsRNA
compound synthesis. The template DNA used for each primer set is also outlined.

| | | Template |
|---|---|---|
| Primer | Sequence (5’-3’) | Used |
| GFP 700 bp | F: | peGFP-C1 |
| | TAATACGACTCACTATAGGGAGAGTGAGCAAGGGCGAG | plasmid |
| | GAGCTG (SEQ ID NO: 1) | |
| | R: | |
| | TAATACGACTCACTATAGGGAGATTACTTGTACAGCTCG | |
| | TCCATGC (SEQ ID NO: 2) | |
| GFP 600 bp | F: | |
| | TAATACGACTCACTATAGGGAGAGTGAGCAAGGGCGAG | |
| | GAGCTG (SEQ ID NO: 1) | |
| | R: | |
| | TAATACGACTCACTATAGGGAGAGGTAGTGGTTGTCGG | |
| | GCAGCAG (SEQ ID NO: 3) | |
| GFP 500 bp | F: | |
| | TAATACGACTCACTATAGGGAGAGTGAGCAAGGGCGAG | |
| | GAGCTG (SEQ ID NO: 1) | |
| | R: | |
| | TAATACGACTCACTATAGGGAGAATCTTGAAGTTCACCT | |
| | TGATGCCG (SEQ ID NO: 4) | |
| GFP 400 bp | F: | |
| | TAATACGACTCACTATAGGGAGAGTGAGCAAGGGCGAG | |
| | GAGCTG (SEQ ID NO: 1) | |
| | R: | |
| | TAATACGACTCACTATAGGGAGAATCTTGAAGTTCACCT | |
| | TGATG (SEQ ID NO: 5) | |
| GFP 300 bp | F: | |
| | TAATACGACTCACTATAGGGAGAGTGAGCAAGGGCGAG | |
| | GAGCTG (SEQ ID NO: 1) | |
| | R: | |
| | TAATACGACTCACTATAGGGAGAGAAGAAGATGGTGCG | |
| | CTCCTG (SEQ ID NO: 6) | |
| GFP 200 bp | F: | |
| | TAATACGACTCACTATAGGGAGAGTGAGCAAGGGCGAG | |
| | GAGCTG (SEQ ID NO: 1) | |
| | R: | |
| | TAATACGACTCACTATAGGGAGACCGTAGGTCAGGGTG | |
| | GTCACG (SEQ ID NO: 7) | |
| GFP 100 bp | F: | |
| | TAATACGACTCACTATAGGGAGAGTGAGCAAGGGCGAG | |
| | GAGCTG (SEQ ID NO: 1) | |
| | R: | |
| | TAATACGACTCACTATAGGGAGACGCCCTCGCCGGACA | |
| | CGCTG (SEQ ID NO: 8) | |
| mCherry | F: | pemCherry- |
| 700 bp | TAATACGACTCACTATAGGGAGAGATAACATGGCCATC | C1 plasmid |
| | ATCAAGG (SEQ ID NO: 9) | |
| | R: | |
| | TAATACGACTCACTATAGGGAGACCGGTGGAGTGGCG | |
| | GCCC (SEQ ID NO: 10) | |
| β-lac 750 bp | F: | pFastBacH |
| | TAATACGACTCACTATAGGGAGATGGGTGCACGAGTGG | TA plasmid |
| | GTTACATCG (SEQ ID NO: 11) | |
| | R: | |
| | TAATACGACTCACTATAGGGAGAGTTACCAATGCTTAAT | |
| | CAGTGAGGC (SEQ ID NO: 12) | |
| Lucif 700 bp | F: | RTG-P1 |
| | TAATACGACTCACTATAGGGAGAGCCTACCGTGGTGTT | cDNA |
| | CGTTT (SEQ ID NO: 13) | |
| | R: | |
| | TAATACGACTCACTATAGGGAGATTACGGTTTATCATCC | |
| | CCCTCGG (SEQ ID NO: 14) | |
| VSV M | F: | cDNA from |
| Protein | TAATACGACTCACTATAGGGAGAGATTCTCGGTCTGAA | VSV |
| | GGGGAAAGG (SEQ ID NO: 15) | infected |
| | R: | cells |
| | TAATACGACTCACTATAGGGAGAGGCTGATAGAATCCA | |
| | GGACCCACG (SEQ ID NO: 16) | |
| VSV N | F: | cDNA from |
| Protein | TAATACGACTCACTATAGGGAGATCTGTTACAGTCAAGA | VSV |
| | GAATCATTG | infected |
| | (SEQ ID NO: 17) | cells |
| | R: | |
| | TAATACGACTCACTATAGGGAGATTGCAGAGGTGTCCA | |
| | AATCT | |
| | (SEQ ID NO: 18) | |
| HCoV-229E | F: | cDNA from |
| RdRp | TAATACGACTCACTATAGGGAGATTATAGTTGCGTCATC | HCoV- |
| | GCCT | 229E |
| | (SEQ ID NO: 19) | infected |
| | R: | cells |
| | TAATACGACTCACTATAGGGAGATTAGGATCGTCAACAT | |
| | CGGC | |
| | (SEQ ID NO: 20) | |
| HCoV-229E | F: | cDNA from |
| Spike | TAATACGACTCACTATAGGGAGAACCTAGCTTGCCCAG | HCoV- |
| Protein | AAGTG | 229E |
| | (SEQ ID NO: 21) | infected |
| | R: | cells |
| | TAATACGACTCACTATAGGGAGAAAGCTGTCTGGAAGC | |
| | ACGAA | |
| | (SEQ ID NO: 22) | |
| HCoV-229E | F: | cDNA from |
| M Protein | TAATACGACTCACTATAGGGAGACCAATCATATATGCAC | HCoV- |
| | ATAGACC | 229E |
| | (SEQ ID NO: 23) | infected |
| | R: | cells |
| | TAATACGACTCACTATAGGGAGAGTCATGTTGCTCATG | |
| | GGAG | |
| | (SEQ ID NO: 24) | |
| HCoV-229E | F: | cDNA from |
| N Protein | TAATACGACTCACTATAGGGAGAGTTGCTGTTGATGGT | HCoV- |
| | GCTAA | 229E |
| | (SEQ ID NO: 25) | infected |
| | R: | cells |
| | TAATACGACTCACTATAGGGAGATACCCAAGTGTGGAT | |
| | GGTCT | |
| | (SEQ ID NO: 26) | |

Impact of dsRNA Exposure on Cell Viability

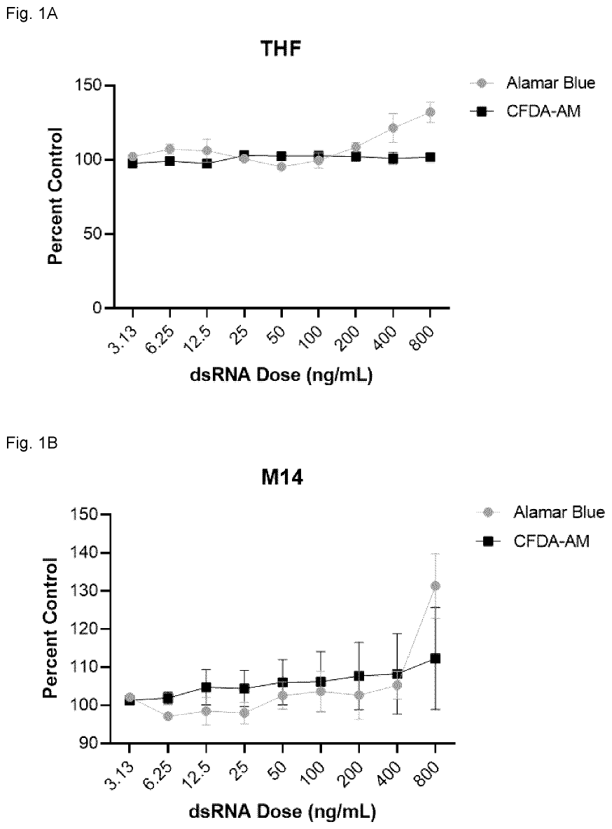

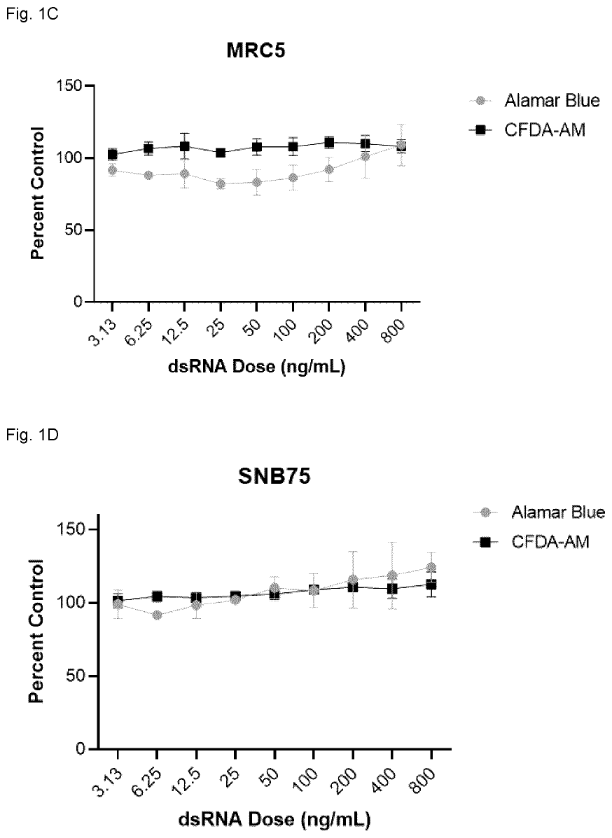

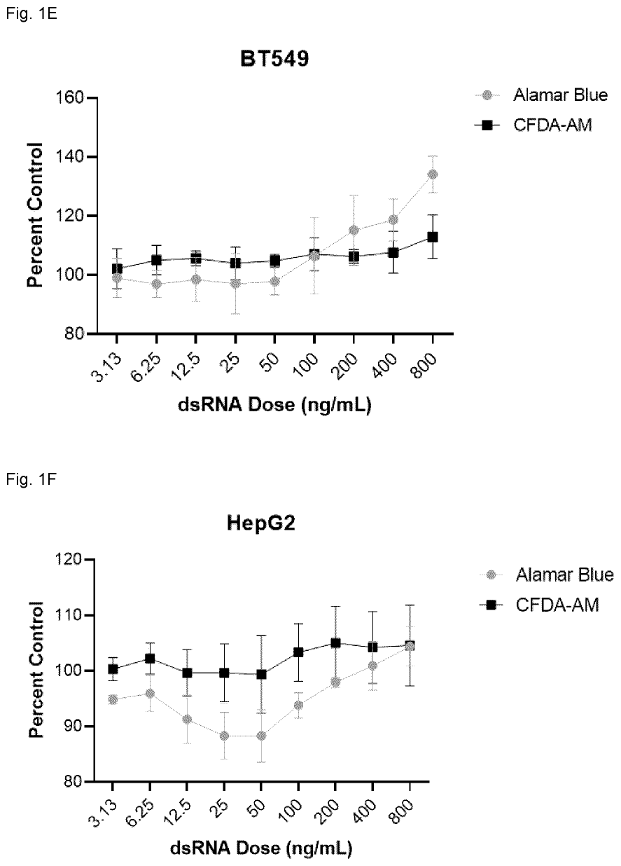

THF (1.0×10⁴ cells/well), M14 (1.5×10⁴ cells/well), MRC5 (1.5×10⁴ cells/well), SNB75 (1.5×10⁴ cells/well), BT549 (1.5×10⁴ cells/well) were seeded in a 96-well tissue culture plate (BD Falcon) and allowed to adhere overnight. Cells were then treated with 700 bp dsRNA compound at varying concentrations (800, 400, 200, 100, 50, 25, 12.5, 6.25, 3.13 ng/mL) diluted in phosphate buffered saline (PBS, Corning) and control wells were exposed to PBS alone. For cells treated with dsRNA compound in a concentration of 100 ng/mL, the number of dsRNA compound molecules per cell is 55,800 molecules. For each dilution, eight replicate wells were used. Following 30 hr of exposure, media was removed and wells were washed once with PBS prior to the addition of Alamar Blue (ThermoFisher) and 5-carboxyfluorescein diacetate acetoxymethyl ester (CFDA-AM, ThermoFisher), two fluorescent cell viability dyes. Alamar Blue measures mitochondrial metabolic activities and CFDA-AM measures cellular membrane integrity. Following a 1 hr incubation in the dark, fluorescence was measured using a BioTek HT Synergy Plate Reader. The plates were read at an excitation of 530 nm and an emission of 590 nm for Alamar Blue as well as an excitation of 485 nm and an emission of 528 nm for CFDA-AM. All relative fluorescent units (RFUs) were normalized to the PBS control, RFUs for each cell type and are reported as percent control. For the six cell lines described, this experimental procedure was repeated three times. Results are shown in FIG. 1A to 1F.

Inhibition of VSV-GFP by the dsRNAi Pathway

### VSV-GFP Inhibition Measured by Plate Reader Assay.

HEL-299 cells were seeded in a 24-well plate at a density of 7.5×10⁴ cells/mL. Following overnight adherence, monolayers were pretreated for 2 hr with 1000 ng/mL of either β-lactamase (mis-matched), or eGFP (matched) sequence specific dsRNA compounds in serum free media. Monolayers were subsequently infected with VSV-GFP (Indiana) at a MOI of 0.1. GFP fluorescence was measured by plate reader when infection of mock-treated cells reached 80% (usually 16-24 hours post-infection). All treated samples were calculated as fold relative to mock treated cells. This experiment was repeated three times.

### VSV-GFP Inhibition as Measured by TCID50/mL

Impact of dsRNA with Different Exposure Times on dsRNAi Inhibition of VSV-GFP

M14 cells were seeded in a 24-well plate (BD Falcon) at a density of 7.5×10⁴ cells/well. Following overnight adherence, the media in all wells was replaced with fresh media. Monolayers were then treated with 500 ng/mL of either mCherry (mis-matched) or eGFP (matched) sequence specific dsRNA compounds (700 bp each) for 5 hr, 4 hr, 3 hr, 2 hr, 1 hr and 0 hr before infection with VSV-GFP at a MOI of 1. After 24 hr of viral infection, supernatants from each well were collected and titred by TCID50 assay as described above. This experiment was repeated three times. Results are shown in FIG. 6.

Impact of dsRNA Length on dsRNAi Inhibition of VSV-GFP

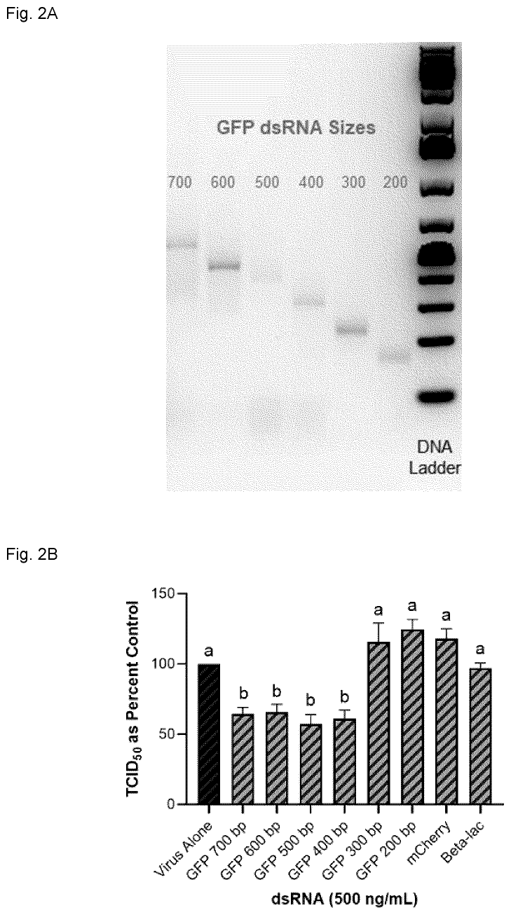

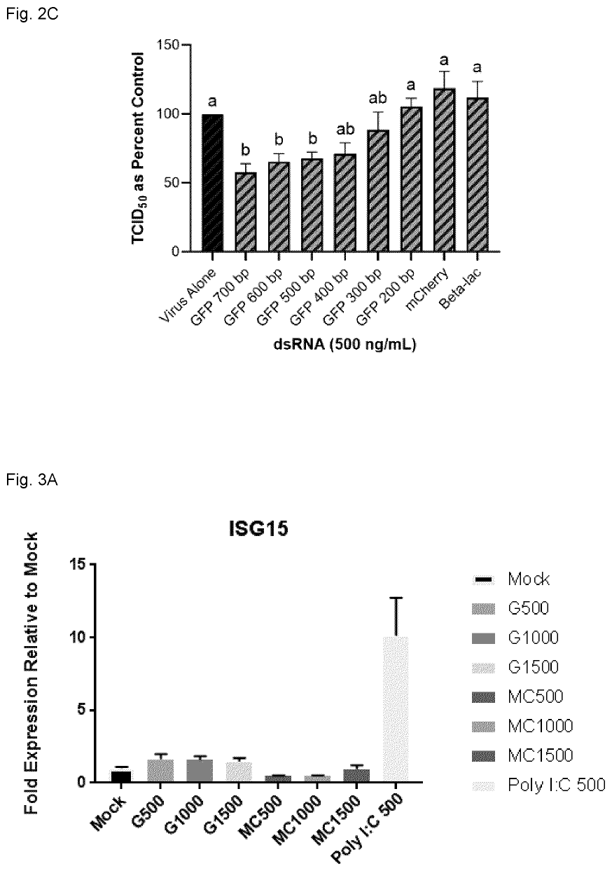

THF and SNB75 cells were seeded in a 24-well plate at a density of 5.0×10⁴ cells/well. Following overnight adherence, the media in all wells was replaced with fresh media. Monolayers were then treated with 500 ng/mL of either 700 bp mCherry (mis-matched), 700 bp p-lac (mis-matched), 700 bp eGFP (matched), 600 bp eGFP (matched), 500 bp eGFP (matched), 400 bp eGFP (matched), 300 bp eGFP (matched) or 200 bp eGFP (matched) for 2 hr before infection with VSV-GFP at a MOI of 0.1. After 24 hr of viral infection, supernatants from each well were collected and titred by TCID50 assay as described above. This experiment was repeated twice. Results are shown in FIGS. 2B and 2C.

Impact of dsRNA Encoding Viral Gene on dsRNAi Inhibition of VSV-GFP

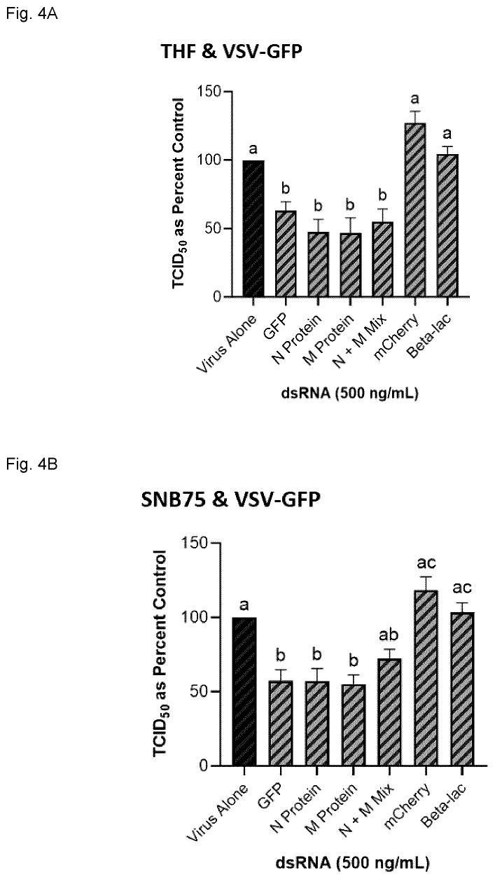

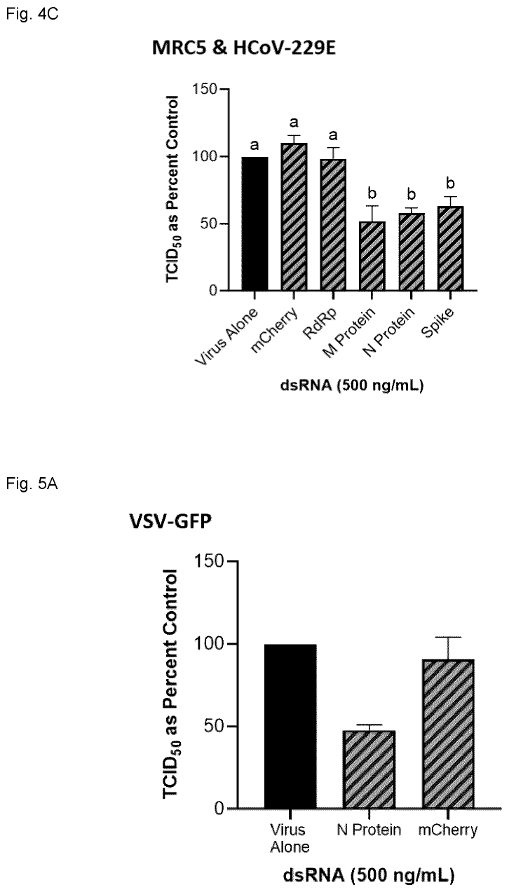

Because eGFP is not naturally present in VSV, dsRNA compounds from the M protein and N protein of the virus were also used to test whether dsRNA compound encoding a natural virus specific gene could also result in dsRNAi viral inhibition. THF and SNB75 cells were seeded in 24-well plates at a density of 5.0×10⁴ cells/well. Following overnight adherence, monolayers were pre-treated for 2 hr with 500 ng/mL of either mCherry (mis-matched), p-lactamase (mis-matched), M VSV gene (matched), N VSV gene (matched), N VSV gene (matched) and M VSV gene (matched) mix, GFP (matched) sequence specific dsRNA compounds in serum free media. Monolayers were subsequently infected with VSV-GFP (Indiana) at a MOI of 0.1. Cell supernatant was collected at 24 hr post-infection and titred by TCID₅₀ assay as described above. Results are shown in FIGS. 4A and 4B.

Viral Inhibition of VSV and HCoV-229E by Sequence Matched dsRNA in pBECs

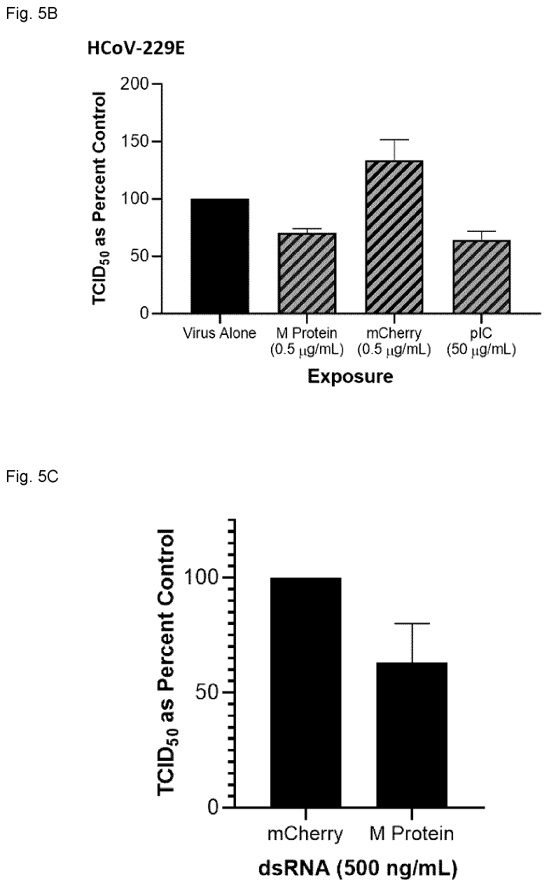

Primary bronchial epithelial cells (pBECs) from a healthy donor were cultured using 1 micron inserts in 24-well companion plates. The pBECs were grown until they reached a confluency of approximately 7.5×10⁴ cells/insert with regular media changes occurring every three days. Once confluent (approximately 30 days), cells were pre-treated for 2 hr with 500 ng/mL of either mCherry (mis-matched) dsRNA compound or N VSV gene (matched) dsRNA compound. Cells were then infected with VSV-GFP (Indiana) at a MOI of 0.1. After 24 hr of viral infection, supernatants from each well were collected and titred by TCID₅₀ assay as described above (n=2). To test whether dsRNA compounds could also inhibit HCoV-229E on pBECs, a separate experiment was performed wherein the cells were pre-treated for 2 hr with 500 ng/mL of either mCherry (mis-matched) dsRNA compound, or M HCoV-229E gene (matched) dsRNA compound or poly I:C (50 μg/mL). Cells were then infected with HCoV-229E at an MOI of 0.1. After 24 hr of viral infection, supernatants from each well were collected and titred by TCID₅₀ assay as described above (n=3). Results are shown in FIGS. 5A and 5B.

To determine whether transfection of long dsRNA compounds could also induce viral inhibition in pBECs, 500 ng/mL of either HCoV-229E M protein dsRNA sequence (matched) or mCherry dsRNA sequence (mis-matched) was transfected directly into pBEC cells using the FuGENE transfection reagent (n=2). Following a 24 h incubation, transfected cells were washed with PBS and infected with HCoV-229E (MOI=0.1) for 24 h before supernatants were collected and used for TCID₅₀ quantification. Results are shown in FIG. 5C.

Long dsRNA Compounds do not Require Transfection to Induce Knockdown while siRNA Molecules do.

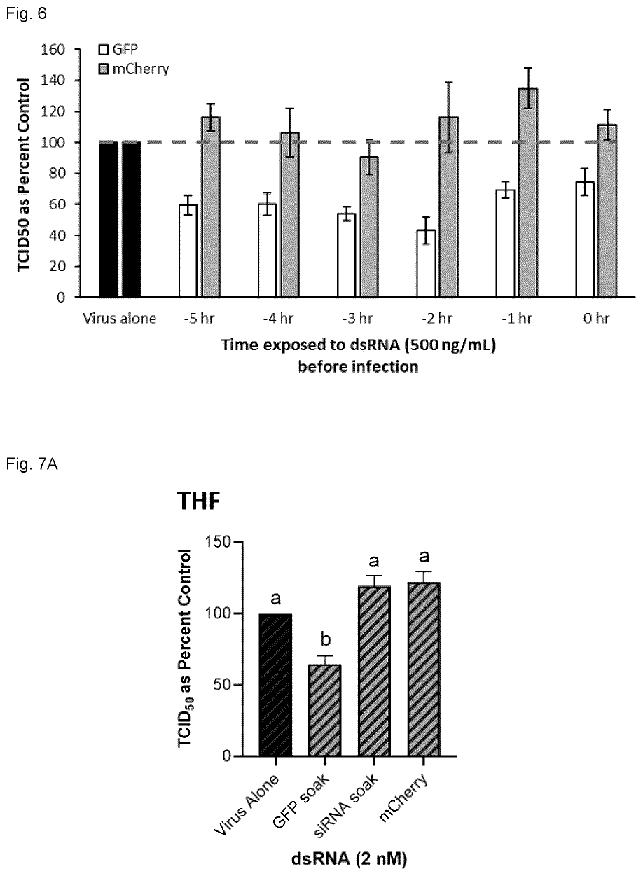

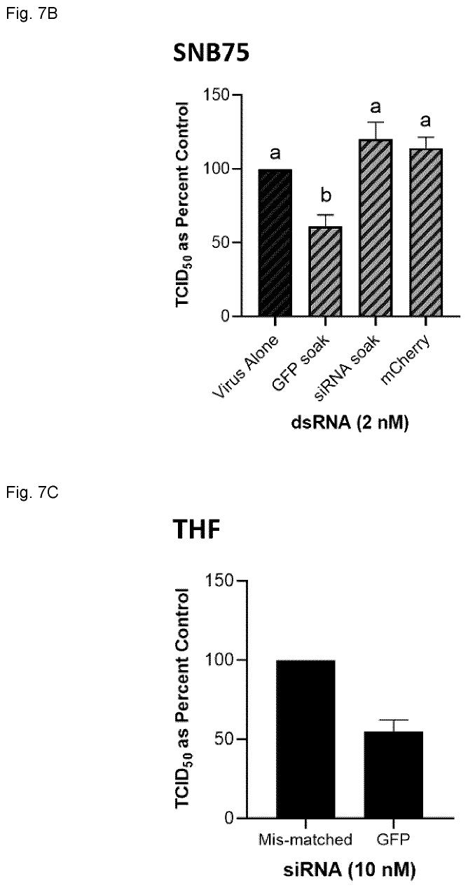

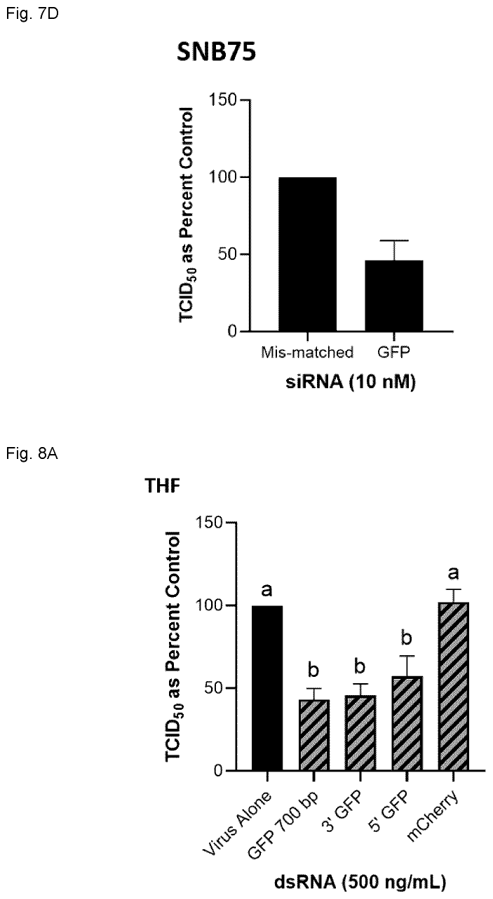

THF and SNB75 cells were seeded in a 24-well plate at a density of 5.0×10⁴ cells/well. Following overnight adherence, the media in all wells was replaced with fresh media. Monolayers were then pre-treated for 2 h with 2 nM of either 700 bp mCherry (mis-matched) dsRNA compound, 700 bp eGFP (matched) dsRNA compound, or GFP siRNA (matched, Ambion Silencer®). Cells were then infected with VSV-GFP (Indiana) at a MOI of 0.1. Following 24 hr of viral infection, supernatants from each well were collected and titred by a TCID₅₀ assay as described above (n=6). Results are shown in FIGS. 7A and 7B.

To confirm that the siRNA molecules used were capable of knockdown but needed transfection to enter cells, THF and SNB75 cells were seeded in a 24-well plate at a density of 5.0×10⁴ cells/well. Following overnight adherence, the media in all wells was replaced with fresh media. Both eGFP siRNA and control siRNA (mis-matched, Ambion Silencer®) were transfected directly into THF and SNB75 cells using lipofectamine. Following a 24 h incubation, transfected cells were washed with PBS and infected with VSV-229E (MOI=0.1) for 24 h before supernatants from each well were collected and used for TCID₅₀ quantification. Results are shown in FIGS. 7C and 7D.

Impact of Sequence Orientation on Long dsRNA Induced Viral Knockdown

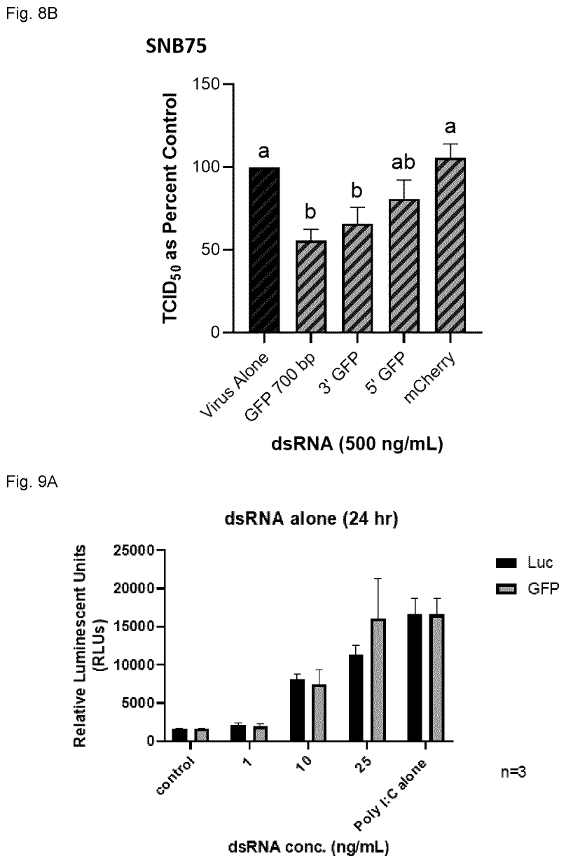

To test whether long dsRNAi had a preference for the 5′ or 3′ ends of molecules, long dsRNA compounds of 900 bp in length were created wherein 800 bp of eGFP sequence was either on the 5′ or 3′ end of the molecule. THF and SNB75 cells were seeded in a 24-well plate at a density of 5.0×10⁴ cells/well. Following overnight adherence, the media in all wells was replaced with fresh media. Monolayers were then pre-treated for 2 hr with 500 ng/mL of either 900 bp dsRNA compounds with GFP on the 5′ end (5′GFP) or GFP on the 3′ end (3′GFP). As controls, additional wells were treated for 2 hr with 700 bp eGFP (positive control) and 700 bp mCherry (negative control). Following the 2 hr pre-treatment, cells were infected with VSV-GFP (Indiana) at an MOI of 0.1. Following 24 hr of viral infection, supernatants from each well were collected and titred by TCID₅₀ assay as described above (n=6). Results are shown in FIGS. 8A and 8B.

Influence of Sequence Matched dsRNA on IFN Inducible Luminescence

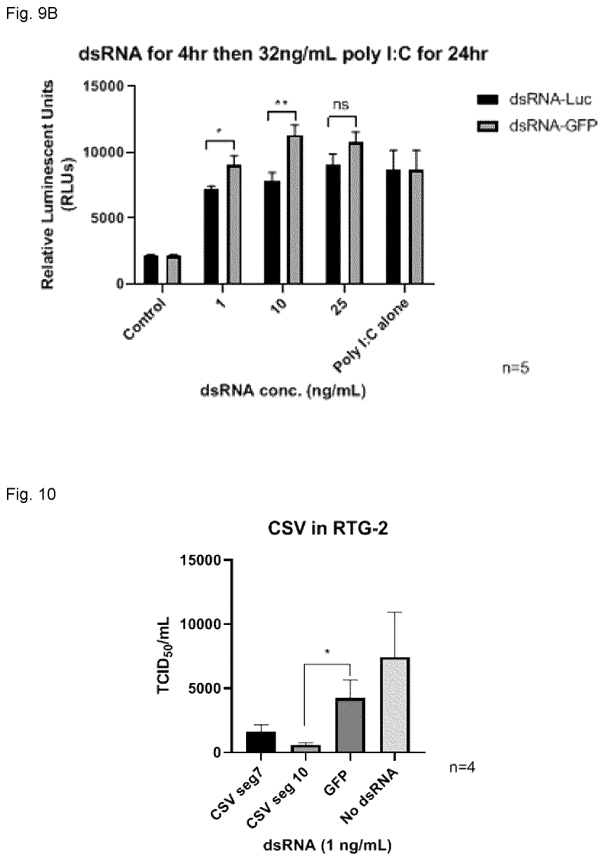

To determine whether desired dsRNA compound doses would stimulate the IFN response and thereby result in luciferase production, RTG-P1 rainbow trout gonadal cells were seeded in 12 well plates at a density of 2.5×10⁵ cells/well. Following overnight adherence, monolayers were treated with 700 bp dsRNA compound encoding either luciferase (Luc, matched) or eGFP (mis-matched) at varying concentrations (1-25 ng/mL) for 24 hr. Using a luciferase assay system (Promega) chemiluminescence was measured on a BioTek HT Synergy Plate Reader. Any luciferase activity would indicate stimulation of the IFN pathway. Results are shown in FIG. 9A.

The aforementioned doses of dsRNA compound were then used to reveal whether they could suppress IFN induced luciferase production when stimulated with poly IC (a potent IFN inducer). RTG-P1 cells were seeded as described above. Following overnight adherence, monolayers were treated with dsRNA compound of either Luc (matched) or eGFP (mis-matched) at 1, 10 and 25 ng/mL for 4 hr prior to treatment with 32 ng/mL of poly IC to stimulate luciferase production. At 24 hr post-exposure to poly IC, luciferase production was measured as described above. Results are shown in FIG. 9B.

Inhibition of the aquatic viral pathogen CSV in RTG-2 using sequence matched dsRNA

RTG-2 rainbow trout cells were seeded in 12-well plates at a density of 2.5×10⁵ cells/well. Following overnight adherence, monolayers were pre-treated for 4 hr with 1 ng/mL of eGFP (mis-matched), CSV segment 7 (matched), or CSV segment 10 (matched) dsRNA compounds in serum free media. All dsRNA compounds were 700 bp in length. Monolayers were subsequently infected with CSV at an MOI of 1 for 4 hr then replaced with 2% FBS media. Cell supernatant was collected at day 7 post-infection and titred by TCID₅₀ assay (n=4). Results are shown in FIG. 10.

Assessing Transcript Levels of ISGs Following Exposure to dsRNA

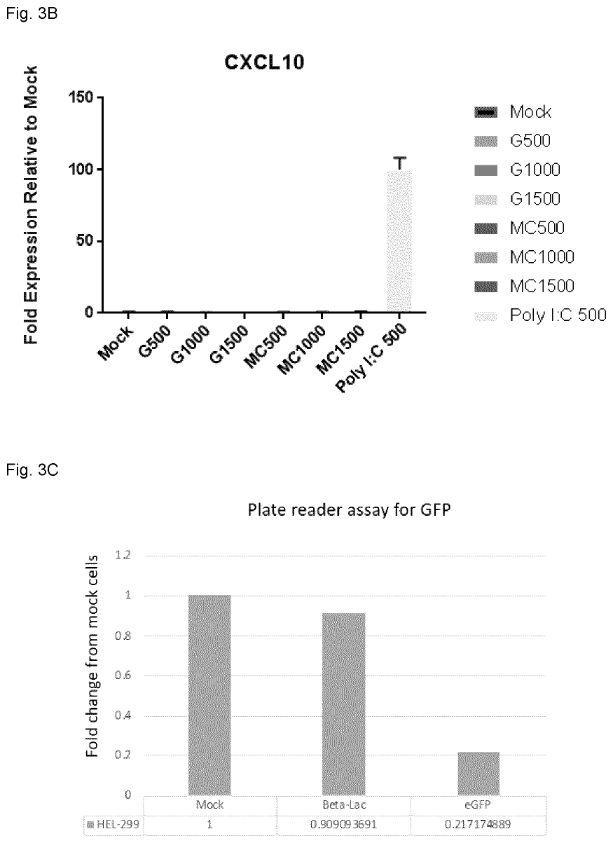

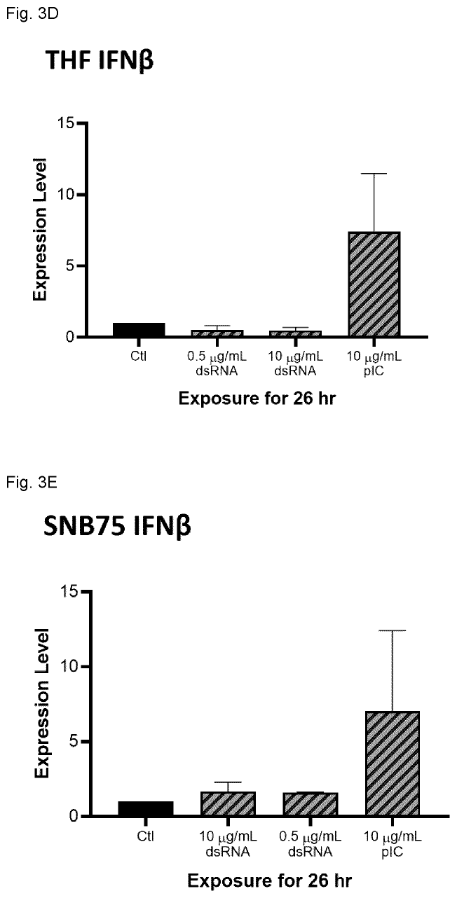

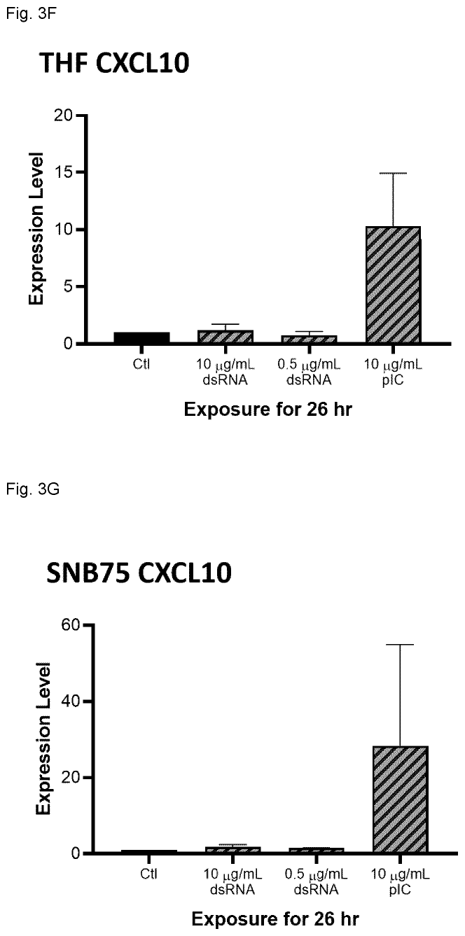

To confirm that the IFN pathway was not being stimulated by the selected doses of dsRNA compounds, transcript levels of ISGs were analyzed following exposure. HEL-299 cells were seeded in a 24-well plate at a density of 7.5×10⁴ cells/mL. Following adherence overnight, monolayers were pre-treated for 2 hr with 500, 1000 or 1500 ng/mL of either mCherry (mis-matched) or eGFP (matched) sequence specific dsRNA compounds, or poly IC (500 ng/mL) in serum free media. At 24 hr post-treatment, total RNA was extracted from test wells by TRIzol™ (ThermoFisher) in accordance with the manufacturer's instructions. To remove any contaminating genomic DNA, RNA samples underwent DNase treatment using the Turbo DNase Kit (ThermoFisher) as outlined by the manufacturer. Complementary DNA (cDNA) was synthesized from 500 ng of total RNA using the iScript cDNA Synthesis Mix (Bio-Rad) in accordance to the manufacturer's instructions. Expression of β-actin, CXCL10 and ISG15 was measured via qRT-PCR. ISG15 and CXCL10 expression was normalized to β-actin. Results are shown in FIGS. 3A and 3B.

Confirming that the Observed Knockdown is Through RNAi

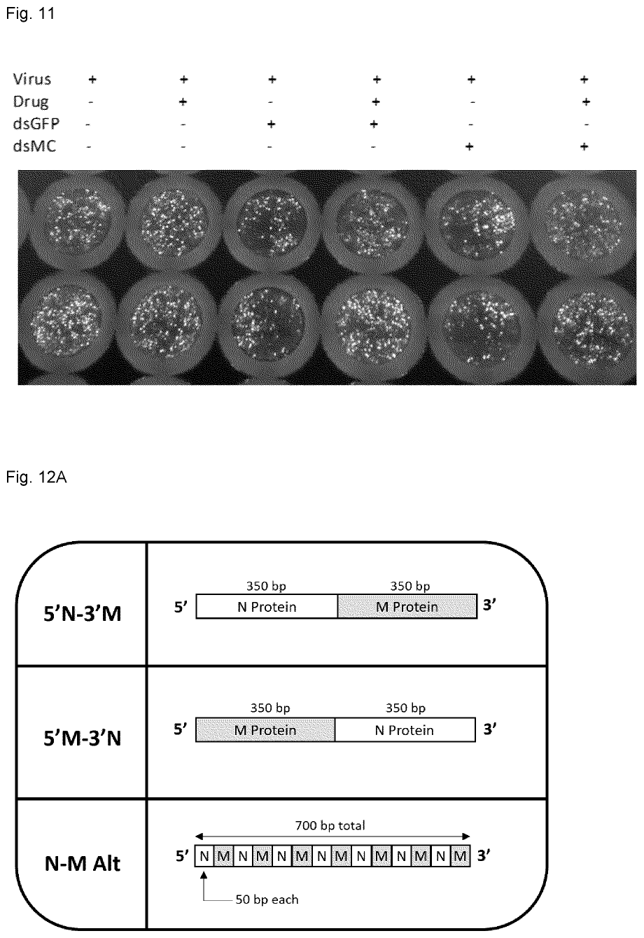

HEL-299 cells were seeded into a 96-well plate at a density of 1.5×10⁴ cells/well. Following overnight adherence, cells were pre-treated for 2 hr with 500 ng/mL of either mCherry (mis-matched), or eGFP (matched) sequence specific dsRNA compounds in serum free media in the presence or absence of 12.5 μM aurintricarboxylic acid (DICER inhibitor). Wells were then subsequently infected with VSV-GFP (Indiana) at a MOI of 0.1. After 24 hr of infection, plates were visualized by fluorescence microscopy. Results are shown in FIG. 11.

Using Synthesized, Combination dsRNA Compounds to Induce Viral Inhibition

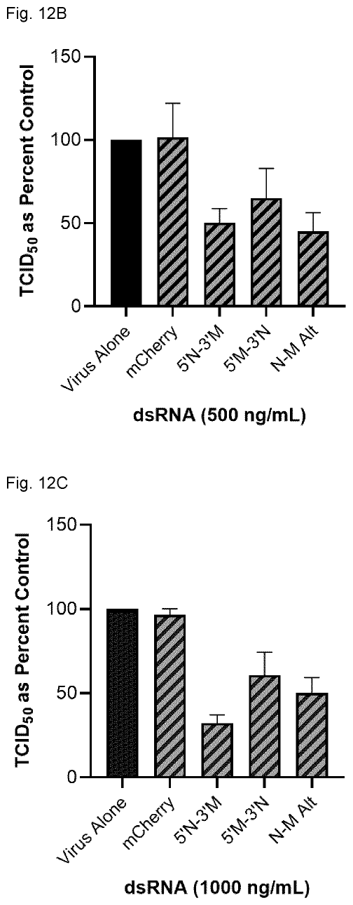

To synthesize dsRNA compounds that contain more than a single VSV viral gene, three gBlock DNA templates were produced that were used to produce 700 bp dsRNA compounds containing different target sequences. The first molecule produced was 5′N-3′M wherein the first 350 bp was a N VSV gene sequence (Genbank Accession No. M15213.1) while the last 350 bp was a M VSV gene sequence (Genbank Accession No. X04452.1). The second molecule produced was 5′M-3′N wherein the first 350 bp was a M VSV gene sequence and the last 350 bp was a N VSV gene sequence. The third molecule is referred to as N-M Alt and contained alternating 50 bp stretches of each gene (first N, then M, etc.) for the entire 700 bp dsRNA sequence. Representative images of these molecules are presented in FIG. 12A. The dsRNA compounds sequences are shown in Table 2 below. Although the dsRNA compounds sequences are represented as DNA, it will be understood that thymidine (T) is replaced by uracil (U) in the sequences.

TABLE 2
dsRNA compound comprising multiple matched sequences

| dsRNA | |
|---|---|
| compound | Sequence |
| 5′M-N3′ | TTCTCGGTCTGAAGGGGAAAGGTAAGAAATCTAAGAAATTAGGGATCGCA |
| | CCACCCCCTTATGAAGAGGACACTAGCATGGAGTATGCTCCGAGCGCTC |
| | CAATTGACAAATCCTATTTTGGAGTTGACGAGATGGACACTCATGATCCG |
| | AATCAATTAAGATATGAGAAATCCTTCTTTACAGTGAAAATGACGGTTAGA |
| | TCTAATCGTCCGTTCAGAACATACTCAGATGTGGCAGCCGCTGTATCCCA |
| | TTGGGATCACATGTACATCGGAATGGCAGGGAAACGTCCCTTCTACAAGA |
| | TCTTGGCTTTTTTGGGTTCTTCTAATCTAAAGGCCACTCCAGCGGTATTGG |
| | ATGGAGTATCGGATGCTTCCAGAACCAGAGCAGATGACAAATGGTTGCCT |
| | TGTATCTACTTGGCTTATACAGAGTGGGCAGAACACAAATGCCTGAATAC |
| | AGAAAAAAGCTCATGGATGGCTGACAAATCAATGCAAAATGATCAATGAA |
| | CAGTTTGAACCTCTTGTGCCAGAAGGTCGTGACATTTTTGATGTGTGGGG |
| | AAATGACAGTAATTACACAAAAATTGTCGCTGCAGTGGACATGTTCTTCCA |
| | CATGTTCAAAAACATGAATGTGCCTCGTTCAGATACGGAACTATTGTTTCC |
| | AGATTCAAAGATTGTGCTGCATTGGCAACATTTGGACACCTCTGCAA |
| | (SEQ ID NO: 27) |
| 5′N-M′3 | TCTGTTACAGTCAAGAGAATCATTGACAACACAGTCATAGTTCCAAAACTT |
| | CCTGCAAATGAGGATCCAGTGGAATACCCGGCAGATTACTTCAGAAAATC |
| | AAAGGAGATTCCTCTTTACATCAATACTACAAAAAGTTTGTCAGATCTAAG |
| | AGGATATGTCTACCAAGGCCTCAAATCCGGAAATGTATCAATCATACATGT |
| | CAACAGCTACTTGTATGGAGCATTGAAGGACATCCGGGGTAAGTTGGATA |
| | AAGATTGGTCAGTTTCGGAATAAACATCGGGAAGGCAGGGGATACAATC |
| | GGAATATTTGACCTTGTATCCTTGAAAGGCCTGGACGGTGTACTTCCAGC |
| | AGATCAAGGTCAACCCGAGTATCACGCTCACTGCGAAGGCAGGGCTTAT |
| | TTGCCACATAGAATGGGGAAGACCCCTCCCATGCTCAATGTACCAGAGCA |
| | CTTCAGAAGACCATTCAATATAGGTCTTTACAAGGGAACGATTGAGCTCA |
| | CAATGACCATCTACGATGATGAGTCACTGGAAGCCGCTCCTATGATCTGG |
| | GATCATTTTAATTCTTCCAAATTTTCTGATTTCAGAGAGAAGGCCTTAATGT |
| | TTGGCCTGATTGTCGAGGAAGAGGCATCTGGAGCTTGGGTCCTGGATTC |
| | TGTCCGCCACTCCAAATGGGCTAGTCTAGCTTCCAGCTTCTGAACAAT |
| | (SEQ ID NO: 28) |
| N-M Alt | TCTGTTACAGTCAAGAGAATCATTGACAACACAGTCATAGTTCCAAAACTT |
| | TCTCGGTCTGAAGGGGAAAGGTAAGAAATCTAAGAAATTAGGGATCGCAT |
| | CCTGCAAATGAGGATCCAGTGGAATACCCGGCAGATTACTTCAGAAAATC |
| | CACCCCCTTATGAAGAGGACACTAGCATGGAGTATGCTCCGAGCGCTCC |
| | CAAAGGAGATTCCTCTTTACATCAATACTACAAAAAGTTTGTCAGATCTAA |
| | ATTGACAAATCCTATTTTGGAGTTGACGAGATGGACACTCATGATCCGAA |
| | GAGGATATGTCTACCAAGGCCTCAAATCCGGAAATGTATCAATCATACAA |
| | TCAATTAAGATATGAGAAATCCTTCTTTACAGTGAAAATGACGGTTAGATG |
| | TCAACAGCTACTTGTATGGAGCATTGAAGGACATCCGGGGTAAGTTGGTC |
| | TAATCGTCCGTTCAGAACATACTCAGATGTGGCAGCCGCTGTATCCCAAT |
| | AAAGATTGGTCAGTTTCGGAATAAACATCGGGAAGGCAGGGGATACAATT |
| | GGGATCACATGTACATCGGAATGGCAGGGAAACGTCCCTTCTACAAGATC |
| | GGAATATTTGACCTTGTATCCTTGAAAGGCCTGGACGGTGTACTTCCATC |
| | TTGGCTTTTTTGGGTTCTTCTAATCTAAAGGCCACTCCAGCGGTATTG |
| | (SEQ ID NO: 29) |

THF cells were seeded in a 24-well plate at a density of 5.0×10⁴ cells/well. Following overnight adherence, the media in all wells was replaced with fresh media. Monolayers were then pre-treated for 2 h with 500 ng/mL or 1000 ng/mL of either 700 bp mCherry (mis-matched), 700 bp 5′N-3′M (matched), 700 bp 5′M-3′N (matched), or 700 bp of N-M Alt dsRNA compounds. Cells were then infected with VSV-GFP at an MOI of 0.1. After 24 hr of viral infection, supernatants from each well were collected and titred by TCID₅₀ assay as described above (n=3). Results are shown in FIGS. 12B and 12C.

### Results

It has been found that dsRNA sequence does not affect IFN production levels therefore the sequence making up the sense and antisense RNA molecules can code for whatever needs to be shut down by RNAi. This can include both host and non-host sequences. To push the dsRNA-mediate response towards RNAi vs. IFN, dsRNA compounds of at least −100 bp are used. dsRNA compounds in the endosome and cytoplasm will induce IFN. dsRNA compounds in the cytoplasm will be cleaved by Dicer and loaded into RISC for RNAi.

Long dsRNA is not toxic given that when assessing the viability of normal or cancer cell lines when treated with 700 base pair long dsRNA compounds, survival was not negatively influenced by the treatment, but higher concentrations of dsRNA enhanced metabolic activity (FIG. 1A-F). 400, 500 and 600 base pair dsRNA compounds were also effective for limiting VSV-GFP when using a matched sequence (GFP), indicating that dsRNA compounds of different length can funnel into RNAi in both normal (THF) and cancer (SNB75) cell lines (FIG. 2A-2C).

While treatment with poly I:C (500 ng/ml) resulted in significant expression of interferon-stimulated genes (ISGs), treatment with dsRNA compounds, matched or mis-matched, up to 1500 ng/ml in HEL 299 cells resulted in no induction of ISGs above base-line, demonstrating that dsRNAi can occur at doses that do not induce ISGs or Type I interferons (FIGS. 3A, 3B and 3D-3G). dsRNA compounds containing matched sequences (GFP, N or M) reduced virus titres compared to control, mis-matched sequences (mCherry, betalac) which did not, demonstrating that dsRNAi can knockdown a viral gene other than GFP (FIG. 4A-4C). dsRNAi can knockdown viruses in human primary Bronchial Epithelial Cells (pBECs) which are polarized, differentiated human airway epithelial cells. Antiviral protection is comparable to poly IC (IFN inducer), with both rhabdoviruses (VSV) and coronaviruses (HCoV-229E) (FIG. 5A-5C). dsRNAi can knockdown virus when introduced before or during virus infection (FIG. 6).

As shown in FIG. 7A-7D, GFP-dsRNA compounds can enter cells without requiring transfection, while GFP-siRNA requires transfection to mediate knockdown. Knockdown of VSV-GFP is comparable between GFP-dsRNA compound administration without transfection reagent vs. GFP-siRNA transfection in both normal and cancer cell lines.

As shown in FIGS. 8A and 8B, long (700 bp) dsRNAi induces viral inhibition whether the matched sequence is on the 3′ or 5′ end of the dsRNA compound. In certain cell types e.g. SNV75, the matched sequence on the 3′ end of the dsRNA compound may be preferable. It was also demonstrated that dsRNAi knockdown is via the RNAi pathway (FIG. 11). It was further found that the 700 bp dsRNA compounds containing multiple matched sequences effectively inhibited viral growth (FIGS. 12A and 12B).

Knockdown using the dsRNA compounds described herein was observed in fish cells as well. As shown in FIGS. 9A and 9B, in fish cells, dsRNAi can knockdown inducible endogenous transcripts even in the presence of low levels of IFN. Doses of 1 ng/mL and 10 ng/mL of 700 bp dsRNA compounds demonstrate a sequence dependent knockdown of luciferase while 25 ng/mL shows a trend but is not statistically significant, suggesting that the dsRNAi effect decreases as IFN production increases. As shown in FIG. 10, matched dsRNA compounds (CSV seg7-dsRNA and CSV seg10-dsRNA) substantially knocked down virus titres in RTG-2 cells however mis-matched sequence (GFP-dsRNA) did not. Knock down was found to be greater in fish cells than what was observed in human cells.

### REFERENCES

Abedini, F., Ebrahimi, M., and Hosseinkhani, H. (2018). Technology of RNA Interference in Advanced Medicine. MicroRNA, 7(2), 74-84.
Buchon N, Vaury C (2005) RNAi: a defensive RNA-silencing against viruses and transposable elements. Heredity 96: 195-202.
DeWitte-Orr S J, Mossman K M (2010) dsRNA and the innate antiviral immune response. Future Virol 5: 325-341.
DeWitte-Orr S J, Collins S E, Bauer C M T, Bowdish D M, Mossman K L (2010) An Accessory to the ‘Trinity’: SR-As Are Essential Pathogen Sensors of Extracellular dsRNA, Mediating Entry and Leading to Subsequent Type I IFN Responses. PLoS Pathog 6(3): e1000829. Fire, A., Xu, S., Montgomery, M. K., Kostas, S. A., Driver, S. E., and Mello, C. C. (1998). Potent and specific genetic interference by double-stranded RNA in Caenorhabditis elegans. Nature, 391(6669), 806-811.
Jacobs B, Langland J (1996) When two strands are better than one: the mediators and modulators of the cellular responses to double-stranded RNA. Virology 219:339-349.
Maillard P V, et al. (2016). Inactivation of the type I interferon pathway reveals long double-stranded RNA-mediated RNA interference in mammalian cells. The EMBO Journal. 35(23), 2505-2518.
Meister G, Tuschl T (2004) Mechanisms of gene silencing by double-stranded RNA. Nature. 431:343-349.
Noyce R S, Collins Se, Mossman K L. (2006) Identification of a novel pathway essential for the immediate-early, interferon-independent antiviral response to enveloped virions. J Virol 80:226-235.

1. A double stranded RNA (dsRNA) compound, optionally a therapeutic dsRNA compound, comprising a guide strand and a passenger strand, the guide strand and the passenger strand each having a length of at least 300 base pairs (bp), the guide strand comprising a segment complementary to a target nucleic acid sequence of a target gene transcript, optionally wherein the length of the guide strand is between about 300 bp and about 3000 bp, and/or the segment complementary to the target nucleic acid sequence has a length of between about 20 bp and about 900 bp.

2.-3. (canceled)

4. The dsRNA compound of claim 1, wherein the segment complementary to the target nucleic acid sequence is proximal to or at the 3′ end of the guide strand or is proximal to or at the 5′ end of the guide strand.

5. (canceled)

6. The dsRNA compound of claim 1, wherein the guide strand comprises one or more further different segment(s), wherein each of the one or more further different segment(s) is complementary to a different target nucleic acid sequence in the same target gene transcript or in different target gene transcripts, optionally wherein one or more segment(s) is proximal to or at the 3′ end of the guide strand and one or more segment(s) is proximal to or at the 5′ end of the guide strand.

7.-8. (canceled)

9. The dsRNA compound of claim 1, wherein the guide strand comprises one or more mismatched sequences non-native to the target nucleic acid sequence and/or the guide strand or the passenger strand comprises an overhang, optionally wherein the overhang is complementary to or corresponds to the target nucleic acid sequence or target gene transcript.

10.-12. (canceled)

13. The dsRNA compound of claim 1, wherein the dsRNA compound comprises one or more chemically modified bases, optionally wherein the chemical modification is selected from 2′Omethyl (2′-O-Me), 2′-O-methoxyethyl (2′O-MOE), 2′fluoro (2′F) and locked nucleic acid monomer (LNAM), or comprises non-modified RNA.

14.-15. (canceled)

16. The dsRNA compound of claim 1, wherein the guide strand and/or the passenger strand comprises a 5′-triphosphate end, a 5′-diphosphate end, or a 5′-monophosphate end and/or wherein the dsRNA compound does not comprise a replication gene, optionally a viral replication gene.

17.-19. (canceled)

20. The dsRNA compound of claim 1, wherein the target gene transcript corresponds to a pathogen gene transcript such as a viral gene transcript, optionally wherein the viral gene transcript is a structural viral gene transcript, optionally encoding a spike (S) protein, a membrane (M) protein or a nucleocapsid (N) protein transcript and/or wherein the viral gene transcript is an influenza virus, coronavirus, rabies, ebolavirus, dengue virus, rotavirus, or rhabdovirus gene transcript.

21.-22. (canceled)

23. The dsRNA compound of claim 20, wherein the coronavirus gene transcript is human coronavirus gene transcript, such as human coronaviruses SARS-CoV-1, SARS-CoV-2, MERS-CoV, HCoV-229E, HCoV-OC43, HCoV-NL63 and HCoV-HKU1 gene transcript.

24. The dsRNA compound of claim 1, wherein the target gene transcript is a disease gene transcript, optionally an oncogene transcript, optionally wherein the disease is cancer or a lymphoproliferative disorder such as plasma cell proliferative disorder.

25. (canceled)

26. The dsRNA compound of claim 1, for use in silencing a target gene transcript in a vertebrate cell or subject comprising the target nucleic acid sequence, and/or for use in reducing replication or infectivity of a pathogen infection in the vertebrate cell or subject, respectively.

27. The dsRNA compound of claim 1, for use in targeting a mixed cell population by silencing a target gene transcript in a first vertebrate cell type comprising the target nucleic acid sequence and inducing a type I interferon (IFN) response in a second vertebrate cell type, optionally wherein the cell population is in a vertebrate subject.

28. A vector comprising an expression cassette encoding one or more transcript(s) that can form the dsRNA compound of claim 1, optionally wherein the expression cassette comprises a polymerase promoter, optionally a T7 polymerase promoter, for producing the guide strand and the passenger strand, and/or the vector is a plasmid, viral construct or virus, optionally wherein the virus is a lentivirus or an adeno associated virus.

29.-31. (canceled)

32. A conjugate comprising the dsRNA compound of claim 1 and one or more polypeptide, such as an antibody, optionally a single chain antibody or a binding fragment, or a label.

33. A composition comprising the dsRNA compound of claim 1, a vector comprising an expression cassette encoding one or more transcript(s) that can form the dsRNA compound or the conjugate comprising the dsRNA compound and one or more polypeptide, such as an antibody, optionally a single chain antibody or a binding fragment, or a label, optionally wherein the concentration of the dsRNA compound is in a dosage amount that is below a selected IFN induction threshold, optionally further comprising a pharmaceutically acceptable carrier and/or lipid transport particles, optionally selected from liposomes, nanoparticles or nanosomes, optionally wherein the composition lacks a delivery mechanism, optionally transport particles.

34.-37. (canceled)

38. A method of silencing a target gene transcript in a vertebrate cell, optionally a disease cell, such as a cancer cell, or subject, optionally wherein the cell or subject is IFN-competent, the method comprising administering to the cell or subject an amount of the dsRNA compound of claim 1, a vector comprising an expression cassette encoding one or more transcript(s) that can form the dsRNA compound, a conjugate comprising the dsRNA compound and one or more polypeptide, such as an antibody, optionally a single chain antibody or a binding fragment, or a label, of or a composition comprising the dsRNA compound, the vector, or the conjugate, optionally further comprising a pharmaceutically acceptable carrier, optionally wherein the cell or subject is an aquatic vertebrate cell or subject such as a fish cell or a fish, optionally a teleost, a terrestrial vertebrate cell or subject such as a bird, or a mammalian cell or subject, optionally a human, cow, pig, horse or sheep cell or subject.

39. The method of claim 38, wherein the amount of the dsRNA, vector, conjugate or composition administered is below a predetermined IFN induction threshold, optionally wherein the predetermined IFN induction threshold is determined using a comparator population of cells, wherein the comparator population of cell is incubated with the dsRNA compound, vector, conjugate or composition and the predetermined IFN induction threshold is determined therefrom.

40.-41. (canceled)

42. The method of claim 38 for reducing replication or infectivity of a pathogen infection in the vertebrate cell or subject, respectively, or for treating a pathogen infection in a subject in need thereof wherein the target nucleic acid sequence corresponds to a pathogen gene transcript sequence, such as a viral gene transcript sequence, optionally wherein the administering is prior to exposure to the pathogen or during exposure to the pathogen.

43.-45. (canceled)

46. The method of claim 42, wherein the pathogen infection has a site of infection and the amount is administered to the site of infection, optionally wherein the amount administered to the site of infection is above an IFN induction threshold, or the amount is administered systemically and the amount is below an IFN induction threshold.

47.-48. (canceled)

49. The method of claim 38, wherein the dsRNA compound, vector, conjugate or composition is administered intranasally, optionally in the form of an aerosol, topically, optionally in the form of a cream, gel, ointment, paste, colloid or suspension, orally, ocularly, rectally, vaginally, by bath, subcutaneously, intravenously, intraperitoneally, intrapleurally, intramuscularly.

50.-57. (canceled)

58. The method of claim 38, wherein the cell is a cancer cell or the subject has cancer and/or wherein the cell is from an immune-privileged site.

59.-60. (canceled)


SEQUENCE LISTING (29 sequences)

SEQ ID NO: 1 - DNA, 44 bp, Artificial Sequence
  Synthetic Construct
taatacgactcactatagggagagtgagcaagggcgaggagctg

SEQ ID NO: 2 - DNA, 46 bp, Artificial Sequence
  Synthetic Construct
taatacgactcactatagggagattacttgtacagctcgtccatgc

SEQ ID NO: 3 - DNA, 45 bp, Artificial Sequence
  Synthetic Construct
taatacgactcactatagggagaggtagtggttgtcgggcagcag

SEQ ID NO: 4 - DNA, 47 bp, Artificial Sequence
  Synthetic Construct
taatacgactcactatagggagaatcttgaagttcaccttgatgccg

SEQ ID NO: 5 - DNA, 44 bp, Artificial Sequence
  Synthetic Construct
taatacgactcactatagggagaatcttgaagttcaccttgatg

SEQ ID NO: 6 - DNA, 44 bp, Artificial Sequence
  Synthetic Construct
taatacgactcactatagggagagaagaagatggtgcgctcctg

SEQ ID NO: 7 - DNA, 44 bp, Artificial Sequence
  Synthetic Construct
taatacgactcactatagggagaccgtaggtcagggtggtcacg

SEQ ID NO: 8 - DNA, 43 bp, Artificial Sequence
  Synthetic Construct
taatacgactcactatagggagacgccctcgccggacacgctg

SEQ ID NO: 9 - DNA, 45 bp, Artificial Sequence
  Synthetic Construct
taatacgactcactatagggagagataacatggccatcatcaagg

SEQ ID NO: 10 - DNA, 41 bp, Artificial Sequence
  Synthetic Construct
taatacgactcactatagggagaccggtggagtggcggccc

SEQ ID NO: 11 - DNA, 47 bp, Artificial Sequence
  Synthetic Construct
taatacgactcactatagggagatgggtgcacgagtgggttacatcg

SEQ ID NO: 12 - DNA, 48 bp, Artificial Sequence
  Synthetic Construct
taatacgactcactatagggagagttaccaatgcttaatcagtgaggc

SEQ ID NO: 13 - DNA, 43 bp, Artificial Sequence
  Synthetic Construct
taatacgactcactatagggagagcctaccgtggtgttcgttt

SEQ ID NO: 14 - DNA, 46 bp, Artificial Sequence
  Synthetic Construct
taatacgactcactatagggagattacggtttatcatccccctcgg

SEQ ID NO: 15 - DNA, 47 bp, Artificial Sequence
  Synthetic Construct
taatacgactcactatagggagagattctcggtctgaaggggaaagg

SEQ ID NO: 16 - DNA, 47 bp, Artificial Sequence
  Synthetic Construct
taatacgactcactatagggagaggctgatagaatccaggacccacg

SEQ ID NO: 17 - DNA, 48 bp, Artificial Sequence
  Synthetic Construct
taatacgactcactatagggagatctgttacagtcaagagaatcattg

SEQ ID NO: 18 - DNA, 43 bp, Artificial Sequence
  Synthetic Construct
taatacgactcactatagggagattgcagaggtgtccaaatct

SEQ ID NO: 19 - DNA, 43 bp, Artificial Sequence
  Synthetic Construct
taatacgactcactatagggagattatagttgcgtcatcgcct

SEQ ID NO: 20 - DNA, 43 bp, Artificial Sequence
  Synthetic Construct
taatacgactcactatagggagattaggatcgtcaacatcggc

SEQ ID NO: 21 - DNA, 43 bp, Artificial Sequence
  Synthetic Construct
taatacgactcactatagggagaacctagcttgcccagaagtg

SEQ ID NO: 22 - DNA, 43 bp, Artificial Sequence
  Synthetic Construct
taatacgactcactatagggagaaagctgtctggaagcacgaa

SEQ ID NO: 23 - DNA, 46 bp, Artificial Sequence
  Synthetic Construct
taatacgactcactatagggagaccaatcatatatgcacatagacc

SEQ ID NO: 24 - DNA, 42 bp, Artificial Sequence
  Synthetic Construct
taatacgactcactatagggagagtcatgttgctcatgggag

SEQ ID NO: 25 - DNA, 43 bp, Artificial Sequence
  Synthetic Construct
taatacgactcactatagggagagttgctgttgatggtgctaa

SEQ ID NO: 26 - DNA, 43 bp, Artificial Sequence
  Synthetic Construct
taatacgactcactatagggagatacccaagtgtggatggtct

SEQ ID NO: 27 - DNA, 700 bp, Artificial Sequence
  Synthetic Construct
ttctcggtctgaaggggaaaggtaagaaatctaagaaattagggatcgcaccacccccttatgaagaggacactagcatggagtatgctccgagcgctccaattgacaaatcctattttggagttgacgagatggacactcatgatccgaatcaattaagatatgagaaatccttctttacagtgaaaatgacggttagatctaatcgtccgttcagaacatactcagatgtggcagccgctgtatcccattgggatcacatgtacatcggaatggcagggaaacgtcccttctacaagatcttggcttttttgggttcttctaatctaaaggccactccagcggtattggatggagtatcggatgcttccagaaccagagcagatgacaaatggttgccttgtatctacttggcttatacagagtgggcagaacacaaatgcctgaatacagaaaaaagctcatggatggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaacatgaatgtgcctcgttcagatacggaactattgtttccagattcaaagattgtgctgcattggcaacatttggacacctctgcaa

SEQ ID NO: 28 - DNA, 700 bp, Artificial Sequence
  Synthetic Construct
tctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattgaaggacatccggggtaagttggataaagattggtcagtttcggaataaacatcgggaaggcaggggatacaatcggaatatttgaccttgtatccttgaaaggcctggacggtgtacttccagcagatcaaggtcaacccgagtatcacgctcactgcgaaggcagggcttatttgccacatagaatggggaagacccctcccatgctcaatgtaccagagcacttcagaagaccattcaatataggtctttacaagggaacgattgagctcacaatgaccatctacgatgatgagtcactggaagccgctcctatgatctgggatcattttaattcttccaaattttctgatttcagagagaaggccttaatgtttggcctgattgtcgaggaagaggcatctggagcttgggtcctggattctgtccgccactccaaatgggctagtctagcttccagcttctgaacaat

SEQ ID NO: 29 - DNA, 700 bp, Artificial Sequence
  Synthetic Construct
tctgttacagtcaagagaatcattgacaacacagtcatagttccaaaactttctcggtctgaaggggaaaggtaagaaatctaagaaattagggatcgcatcctgcaaatgaggatccagtggaatacccggcagattacttcagaaaatccacccccttatgaagaggacactagcatggagtatgctccgagcgctcccaaaggagattcctctttacatcaatactacaaaaagtttgtcagatctaaattgacaaatcctattttggagttgacgagatggacactcatgatccgaagaggatatgtctaccaaggcctcaaatccggaaatgtatcaatcatacaatcaattaagatatgagaaatccttctttacagtgaaaatgacggttagatgtcaacagctacttgtatggagcattgaaggacatccggggtaagttggtctaatcgtccgttcagaacatactcagatgtggcagccgctgtatcccaataaagattggtcagtttcggaataaacatcgggaaggcaggggatacaattgggatcacatgtacatcggaatggcagggaaacgtcccttctacaagatcggaatatttgaccttgtatccttgaaaggcctggacggtgtacttccatcttggcttttttgggttcttctaatctaaaggccactccagcggtattg